SEQUENCE LISTING (3 sequences)

SEQ ID NO: 1 - protein, 13 aa, Homo sapiens
IAGFKGEQGPKGE

SEQ ID NO: 2 - protein, 8 aa, Homo sapiens
KNIVTPRT

SEQ ID NO: 3 - protein, 15 aa, Homo sapiens
GDKVSFFCKNKEKKC

(19) United States
(12) Patent Application Publication
(10) Pub. No.: US 2023/0226105 A1
(43) Pub. Date: Jul. 20, 2023

(54) METHODS AND COMPOSITIONS FOR TREATING CANCER

(71) Applicant: ReversPAH LLC, Denver, CO (US)

(72) Inventors: Norbert F. VOELKEL, Denver, CO (US); Charles MAGOLSKE, Denver, CO (US); Stephen DORDUNOO, Halethorpe, MD (US); Johannes KHINAST, Graz (AT); Erik EGLITE, Lake Forest, IL (US)

(73) Assignee: ReversPAH LLC, Denver, CO (US)

(21) Appl. No.: 18/054,213

(22) Filed: Nov. 10, 2022

Related U.S. Application Data
(60) Provisional application No. 63/277,775, filed on Nov. 10, 2021.

(51) Int. Cl. A61K 33/24; A61B 5/00; A61K 31/495; A61K 31/723

(52) U.S. Cl. CPC A61K 33/24; A61B 5/4848; A61K 31/495; A61K 31/723

(57) ABSTRACT

Pharmaceutical compositions containing tetrathiomolybdate (TTM) are disclosed. Pharmaceutical compositions and formulations that contain TTM along with other co-drugs, such as diethylcarbamazine (DEC) and astaxanthin (ATX), are also disclosed. Formulations include a delayed release oral form that releases the TTM in the gastrointestinal tract after the oral form passes the stomach, and an enteric oral form that is not a delayed release form are disclosed. Methods of treating cancer, treating cancer patients as an adjuvant therapy, and treating pulmonary arterial hypertension by administering the pharmaceutical compositions are further disclosed.

### FIELD

The present disclosure relates to methods and compositions for treating cancer, including adjuvant cancer therapy, that include administering to a patient a copper chelator containing tetrathiomolybdate (TTM) and one or more of diethylcarbamazine (DEC) and astaxanthin (ATX).

### STATEMENT REGARDING SEQUENCE LISTING

The instant application contains a Sequence Listing which is hereby incorporated by reference in its entirety. The XML formatted file, created Nov. 9, 2022, is named Sequence Listing and is 3.1 kbytes in size.

### BACKGROUND

Copper is an essential trace element for eukaryotes, including humans. It plays an important role in critical biological functions such as enzyme activity, oxygen transport and cell signaling. However, due to its high redox activity (a process in which one substance or molecule is reduced and another oxidized; oxidation and reduction being considered together as complementary, processes) and its ability to catalyze the production of free radicals, copper can have a detrimental effect as well, such as on lipids, proteins, DNA and other biomolecules. Particularly, mitochondria are thought to be the major targets for oxidative damage resulting from copper toxicity.

Copper can also interfere with proteins and can displace other metals such as zinc from metalloproteins, thereby inhibiting their proper function. In order to prevent copper from exerting its potentially toxic effects, it usually does not exist in free form, but only as a complex. In the human body, approximately 95% of the copper in plasma is bound to proteins like albumin and ceruloplasmin. Due to its role in metabolism, any imbalance in copper bioavailability inevitably leads to deficiency or toxicity, and all organisms have evolved mechanisms that regulate its absorption, excretion and bioavailability. In mammals, copper absorption occurs in the small intestine via enterocyte uptake, followed by its transfer into the blood by the copper transporter ATP7A.

The liver plays a critical role in copper metabolism, serving both as the site of copper storage and regulating its distribution to serum and tissues and excretion of excess copper into the bile. Particularly, hepatocytes transport and regulate physiological copper via the specialized transporter ATP7B.

Over the past two decades, evidence has shown that copper plays an important role in inflammation and the growth of tumors. The copper concentration in serum and tumor tissue is significantly higher than that of healthy subjects. Blood vessels function to supply nutrients and oxygen and are fundamental to the survival and development of tumors. The growth of tumors relies on the formation of new blood vessels. There are two different ways to form new blood vessels: one is the development of existing vessels and the other is to develop new vessels from endothelial cell precursors angioblasts. There is overwhelming evidence that copper is an important angiogenic factor in tumors. Results on a comparative study of trace elements in normal and tumor tissues showed that the average copper concentration of cancerous tissues is significantly different from the average of normal tissue. Evidence shows that copper can stimulate the proliferation and migration of endothelial cells. Vascular endothelial growth factor (VEGF) is another critical factor in angiogenesis. The high level of VEGF reduced the effect of endocrine therapy and radiotherapy of breast cancer. Some effective treatment methods targeting VEGF have been developed.

The aim of copper depletion therapy is to decrease copper to a level where the signaling pathways of cytokines can be reduced but copper deficiency does not occur. For different types of cancers, the mechanisms required for angiogenesis at the primary and metastatic sites are different. These processes depend on the imbalance between angiogenesis activators and inhibitors. Therefore, the development of an antiangiogenic therapy that would affect multiple activators of angiogenesis is desirable. Compounds that reduce copper levels seem to meet this requirement because copper is a required cofactor of several key angiogenic factors. (Wang et al., Curr Med Chem. 2010; 17(25): 2685-2698).

Tetrathiomolybdate (TTM) and salts thereof, such as ammonium tetrathiomolybdate (ATTM), have been studied for the treatment of cancer and other diseases, such as pulmonary arterial hypertension (PAH). Examples of particular anti-cancer agents include those typically used in chemotherapy for metastatic disease and drugs such as TTM, a copper chelator with anti-inflammatory properties, wherein the amounts of the active agent together with the amounts of the combination anticancer agents have shown in various studies to have an effect in treating various cancers, including, but not limited to, colorectal cancer, head and neck and breast cancer.

In addition to therapies which target immune modulatory receptors affected by tumor-mediated escape mechanisms and immune suppression, there are therapies which target the tumor environment. Angiogenesis inhibitors inhibit the extensive growth of blood vessels (angiogenesis) that tumors require to survive. The angiogenesis promoted by tumor cells to meet their increasing nutrient and oxygen demands for example can be blocked by targeting different molecules.

The use of TTM in cancer studies is well-published and a core treatment issue is regulating the copper levels in the patient. This is accomplished by measuring ceruloplasmin (a major copper-carrying protein) levels in the serum and then increasing or decreasing the amount of TTM based on those levels. Oral doses of TTM, when administered, sends all the TTM to the blood, while the actual copper levels increase or decrease depending on the amount and timing of the administered TTM.

It is desirable to (1) provide protection of TTM from the stomach acid and (2) regulate the amount of TTM released from a tablet or capsule over time, providing improved dosage management and an improved balancing of the TTM administered and the copper levels in the blood. An advantage of a controlled-release TTM formulation is a smoother, more constant plasma concentration and the possibility of once-a-day dosing. Once-a-day dosing is important to patient care and compliance, as taking multiple dosages during the day is often missed due to patients' schedules and forgetfulness, reducing the efficacy of the treatment. It is also desirable to utilize a number of co-drugs, such as diethylcarbamazine (DEC), which greatly enhances the efficacy of TTM, and/or astaxanthin (ATX), in the use for cancer treatment.

Of note, the pathobiology of severe forms of PAH, as worked out over the last decades to a large extent by Dr. Norbert Voelkel, is one of an initial injury to the lung arterial vessels which is followed by proliferation of phenotypically altered pulmonary vascular cells and participation of inflammatory cells and bone marrow-derived cells. Dr. Voelkel has found there is a large overlap between cancer cell biology, including but not limited to lung cancer, and angioobliterative pulmonary vascular disease, such as PAH.

The combination of TTM and DEC (a 5-lipoxygenase inhibitor) has been contemplated as a treatment for PAH. ATX is a keto-carotenoid with antioxidant activity that protects against DNA and mitochondrial damage. ATX attaches to both the outer and inner surfaces of cell membranes, and it also binds to mitochondrial membranes, which explains it's powerful antioxidant activity. Further, ATX protects against lung tissue damage by inhibiting protease activation. As an anticancer agent, ATX exerts its effects partially by inducing Nrf2-dependent signaling. It has been demonstrated that ATX inhibits, to a degree, prostate and colon cancer cell growth, but not enough to be considered an effective therapy for these diseases, when used alone.

Some key concepts describing PAH that are similar and applicable to cancer are cell growth of apoptosis-resistant abnormal cells, inflammation, angiogenesis, and bone marrow-derived stem (precursor cells). In recent years, a pathophysiological concept of vasoconstriction has given room to a cell pathobiology concept of growth of abnormal, quasi-malignant cells. It follows that innovative and disease modifying therapies for cancer need to target ‘angiogenesis’ and inflammation.

ATX's activity as a co-drug with TTM, or with TTM and DEC, adds the inhibition of proteases and a powerful oxidant that protects against DNA and mitochondrial damage. ATX is a carotene that can be extracted from green algae (algae that turn from green to bright red as astaxanthin accumulates) including Haematococcus pluvialis. The ATX extract from algae often contains additional oleoresins that primarily include largely fatty acids. ATX in a more pure and higher concentrated form can also be made from petrochemicals. ATX may also be produced from yeast by fermentation processes.

Sulforaphane (SFN), a dietary component abundant in broccoli and its sprouts, inhibits to a degree malignant cell proliferation and tumor sphere formation of cancer stem-like cells (CSC) in triple-negative breast cancer (TNBC). Analysis of gene expression in TNBC tumor cells revealed that SFN decreases the expression of cancer-specific markers CR1, CRIPTO-3/TDGF1P3 (CR3, a homologue of CR1), and various stem cell markers including Nanog, Aldehyde Dehydrogenase 1A1 (ALDH1A1), Wnt3, and Notch4. Studies have shown SFN may control the malignant proliferation of CSCs in TNBC via Cripto-mediated pathway by either suppressing its expression and/or by inhibiting Cripto/Alk4 protein complex formation. (Castro N P et al., Cancer Prev Res (2019) 12(3):147-158). However trials with SFN have not shown enough efficacy to become a compelling treatment for cancer.

SFN exerts its anticancer effects partially by inducing Nrf2-dependent signaling. It has been demonstrated that silencing of NMRAL2P could protect against SFN-mediated inhibition of cell growth, colony formation, and migration in cancer, including colon cancer. This includes seven monoaza racemate analogues of SFN (SF85 and SF101) or its sulfone (SF86, SF102, SF113, SF134, and SF135), which differ from the parent compound by formal substitutions of the sulfinyl (S═O) group by either sulfimidoyl (S(NR)) or sulfoximidoyl (S(O)(NR)) moieties. The R substituent at the nitrogen is acetyl (as in SF85 with S(NAc) and SF86 with S(O)(NAc)), pentafluorobenzoyl (as in SF101 with S(NC(O)C₆F₅) and SF102 with S(O)(NC(O)C₆F₅)), methyl (as in SF113 with S(O)(NMe)), trifluoroacetyl (as in SF134 with S(O)(NCOCF₃)), and carbamoyl (as in SF135 with S(O)(NCONH₂)).

A principal mechanism of action for the anti-inflammatory component of SFN is the activation of the transcription factor NRf2. This transcription factor is a switchboard that transcribes a host of antioxidant enzyme genes—resulting in the production of antioxidant enzymes, such as superoxide dismutase and catalase. Because inflammation is associated with oxidant stress, SFN reduces the oxidant stress component of inflammation, a goal in cancer treatment. While SFN has shown these effects, SFN is not effective enough to be a drug to treat cancer.

Another co-drug is tert-Butylhydroquinone (TBHQ). Like SFN, the activity of TBHQ includes the inhibition of pSTAT3, important in controlling cancer metastases, and up-regulation of Nrf2, a therapeutic target associated with a broad range of diseases which are characterized by excessive oxidative stress and inflammation and inhibition of SHP2, a target that has relevance in a number of solid tumors and hematological cancers. TBHQ ameliorates doxorubicin-induced cardiotoxicity by activating Nrf2 and inducing the expression of its target genes. (Wang et al., Am J Transl Res. 2015; 7(10): 1724-1735). Its clinical usage and efficacy have the disadvantage of being limited by a dose-dependent cardiotoxicity, and for this reason is not considered as a standard of care for cancer.

Bestatin, an inhibitor of the enzyme leukotriene A4 (LTA4)-hydrolase and also an aminopeptidase inhibitor, inhibits the production of the lung endothelial cell damaging LTB4, and aminopeptidase inhibition has shown some effect in cancer treatment trials, however modest.

Montelukast is an inhibitor of the leukotriene C4 receptor which has been approved and is in use for the treatment of allergic rhinitis. Montelukast, applied to in vitro cell culture experiments has been shown to decrease breast cancer cell growth to a degree, but so far not compelling enough to be a standard of care.

Inhibitors of leukotriene B4 receptors (BLT1, BLT2) include LY293111 and BAY-u9773. LY293111 is a LTB4 receptor inhibitor and reportedly induces cell cycle arrest in large-cell lymphoma cells. There are several reports showing that LY293111 inhibits in cell culture studies the growth of pancreas cancer cells and also inhibits the growth of myeloic leukemia cells. BAY-u9773 is a peptidoleukotriene receptor antagonist that blocks vascular permeability in animal model studies.

Multikine (MK) is a mixture of biologically active, natural cytokines, for which preliminary evidence from studies to date suggests the potential to simulate the body's healthy immune response. These are a combination of molecules and proteins (interleukins, interferons, chemokines, and colony stimulating factors) derived from the stimulation in the culture of normal immune system cells. MK is presently administered as a pre-treatment for head and neck cancer patients.

The proteins expressed on the surface of a cancerous cell can act as antigens for an immune response. However, most of the proteins expressed on the surface of cancerous cells are non-mutated self-antigens. Self-antigens are usually ineffective in triggering an immune response since they are present in healthy as well as cancerous cells. Even in situations where a cancer cell is expressing a mutated protein, antigenic changes in cancerous cells that are created by individual point mutations may be too subtle from the standpoint of the immune system to trigger a significant immune response. Since cancer cells utilize essentially the same cellular proteins as healthy cells, cancer cells can often grow and survive without generating an anti-cancer immune response.

Peptides can have sufficient structure to be recognized with specificity by immunoproteins, such as antibodies, and by immune cells. That is, short peptides having from about 8 to about 30 amino acid residues can have sufficient structure to bind to antibodies and serve as an antigen, epitope or other ligand for proteins involved in the activation of the immune system. However, short peptides often generate no or only a weak immune response when administered alone to a human or animal subject. Often, it is necessary to link or to introduce short peptides with larger proteins or biomolecules to serve as a carrier or an adjuvant to induce an immune response that will generate antibodies specific to the short peptide and to initiate an immune response to these short peptides.

There is a need for peptide-based immunomodulators having a well-defined immunogen to treat cancer that facilitates the generation of a safe and predictable anti-tumor response rather than a mixed response including an immune response to a carrier. There is also a need for the development of peptide-based immunomodulators, and the related need for the identification of peptides capable of being recognized by specific components of the immune systems and generating a specific type of directed immune response.

Peptide constructs based on a Ligand Epitope Antigen Presentation System (LEAPS) peptide heteroconjugates are useful as immunomodulators for modulating the immune response to an autoimmune condition. The LEAPS peptide heteroconjugates are useful for treatment of cancers and localization of these LEAPS heteroconjugate-activated dendritic cells (DCs) at the site of cancer tumors and clusters of cancer cells. The DCs can be labeled to detect or visualize the site of the ongoing disease or cancer in the body of a subject.

The LEAPS peptide heteroconjugates have a protein sequence that binds to a specific class or subclass of immune cells and a protein sequence corresponding with an antigen. The LEAPS peptide heteroconjugates can be used to directly modulate the response of the immune system or specific immune cells to the antigen sequence.

Various antigens associated with autoimmune conditions, often with defined epitopes recognized for some Human Leukocyte Antigens (HLA) genotypes, have been identified, including those associated with Insulin Dependent Diabetes Mellitis (IDDM), Rheumatoid Arthritis (RA) (e.g., collagen type II 390-402 IAGFKGEQGPKGE; SEQ ID NO: 1), Systemic Lupus Erythematousis (SLE), Ankyosing Spondylitis (AS), Pemphius Vulgaris (PV) (epidermal cell adhesion molecule desmoglein 190-204), Multiple Sclerosis (MS), Myelinproteolipid (MPL) (peptide sequence KNIVTPRT; SEQ ID NO: 2), certain types of psoriasis, and uveoretintis (Hammer et al., HLA class I peptide binding specificity and autoimmunity, Adv. Immunol, (1997) 66:67; Tisch et al., Induction of Glutamic Acid Decarboxylase 65-Specific Th2 Cells and Suppression of Autoimmune Diabetes at Late Stages of Disease Is Epitope Dependent, J. Immunol. (1999) 163:1178; Yoon et al., Control of Autoimmune Diabetes in NOD Mice by GAD Expression or Suppression in beta Cells, Science (1999) 284:1183; Ruiz et al., Suppressive Immunization with DNA Encoding a Self-Peptide Prevents Autoimmune Disease: Modulation of T Cell Costimulation, J. Immunol., (1999) 162:3336; Kreo et al., Identification of T Cell Determinants on Human Type II Collagen Recognized by HLA-DQ8 and HLA-DQ6 Transgenic Mice, J. Immunol, (1999) 163:1661). In other cases, peptides are known that induce in animals a condition similar to ones found in humans, such as GDKVSFFCKNKEKKC (SEQ ID NO: 3) for antiphospholipid antibodies associated with thrombosis (Gharavi et al., GDKV-Induced Antiphospholipid Antibodies Enhance Thrombosis and Activate Endothelial Cells In Vivo and In Vitro, J. Immunol., (1999) 163:2922) or myelin peptides for experimental autoimmune encephalitis (EAE) as a model for MS (Ruiz et al., supra.; Araga et al., A Complementary Peptide Vaccine That Induces T Cell Anergy and Prevents Experimental Allergic Neuritis in Lewis Rats, J. Immunol., (1999) 163:476-482; Karin et al., Short Peptide-Based Tolerogens Without Self-Antigenic or pathogenic Activity Reverse Autoimmune Disease, J. Immunol, (1999) 160:5188; Howard et al., Mechanisms of immunotherapeutic intervention by anti-CD40L (CD154) antibody in an animal model of multiple sclerosis, (1999) J. Clin Invest., 103:281).

Moreover, glutamic acid decarboxylase (GAD) and specific peptides have been identified associated with insulin dependent diabetes mellitus (IDDM) (Tisch et al., supra; Yoon et al., supra). Many of these conditions are also characterized by elevated levels of one or more different cytokines and other effectors such as Tumor Necrosis Factor (TNF) (Kleinau et al., Importance of CD23 for Collagen-Induced Arthritis: Delayed Onset and Reduced Severity in CD23-Deficient Mice, J. Immunol. (1999) 162:4266; Preckel et al., Partial agonism and independent modulation of T cell receptor and CD8 in hapten-specific cytotoxic T cells, Eur. J. Immunol. (1998) 28:3706; Wooley et al., Influence of a recombinant human soluble tumor necrosis factor receptor FC fusion protein on type II collagen-induced arthritis in mice, J. Immunol., (1993) 151:6602) as well as auto-antibodies, including in some cases, anti-costimulator molecules, in particular, those for Cytotoxic T-lymphocyte-Associated protein 4 (CTLA-4, CD152) on CD4+ cells (Matsul et al., Autoantibodies to T Cell Costimulatory Molecules in Systemic Autoimmune Diseases, (1999) J. Immunol., 162:4328).

Lung cancer is one of the most common and fatal malignant tumors in the world. The tumor microenvironment (TME) is closely related to the occurrence and development of lung cancer, in which the inflammatory microenvironment plays an important role Inflammatory cells and inflammatory factors in the TME promote the activation of the NF-kappaB and STAT3 inflammatory pathways and the occurrence, development, and metastasis of lung cancer by promoting immune escape, tumor angiogenesis, epithelial-mesenchymal transition, apoptosis, and other mechanisms. Clinical and epidemiological studies have also shown a strong relationship among chronic infection, inflammation, inflammatory microenvironment, and lung cancer.

Inflammation can cause lung tissue damage. During inflammation, the cell division rate, DNA damage, and cell mutation rate in lung tissue are increased. In addition, inflammation increases the likelihood of lung cancer by acting as an initiator or promoter of antiapoptotic signals. It can also cause angiogenesis and provide nutrients for the growth and spread of tumor cells.

Pneumonitis is inflammation of the lungs. It can be caused by breathing in a toxin or allergen, the tumor itself, radiation treatment to the chest, and treatment with certain medications such as chemotherapy. Reduction in pneumonitis and radiation pneumonitis would be a great attribute as adjunct therapy for patients undergoing both radiation therapy and chemotherapy.

### SUMMARY

The present inventors have discovered and reasoned that combining TTM with DEC, or with other co-drugs, such as ATX, or active agents selected from inhibitors of the 5-lipoxygenase enzyme (5-LO), such as DEC or zileuton, inhibitors of the LTA4 hydrolase, inhibitors of LT receptors, SFN, tert-butylhydroquinone, Bestatin, or Montelukast, in concert can have a formidable anti-cancer effect. The present disclosure is directed to methods of treating cancer in a patient in need thereof by administering to the patient a therapeutically effective amount of a copper chelator that includes TTM. The present disclosure is further directed to methods of treating cancer in a patient in need thereof by administering to the patient a therapeutically effective amount of a copper chelator that includes TTM, in combination with one or more of DEC and ATX and/or other co-drugs.

The present inventors have also discovered and reasoned that combining TTM with DEC, or with other do-drugs such as ATX, in concert can dramatically reduce inflammation, pneumonitis, and the inflammatory process, along with pathological manifestations thereof, thereby improving the quality of life and treatment outcomes of cancer patients. Such adjuvant therapy can alleviate inflammation in the alveoli and vasculature of a lung, in lung cancer patients in particular. The present disclosure is further directed to adjuvant cancer therapy and methods of reducing inflammation and pneumonitis in patients with cancer undergoing cancer therapy by administering to the patient a therapeutically effective amount of a copper chelator that that includes TTM, in combination with one or more of DEC and ATX.

The present inventors have also discovered and reasoned that combining TTM with DEC, or with other co-drugs such as ATX, in concert can have a formidable anti-PAH effect. The present disclosure is directed to methods of treating PAH in a patient in need thereof by administering to the patient a therapeutically effective amount of a copper chelator that includes TTM. The present disclosure is further directed to methods of treating PAH in a patient in need thereof by administering to the patient a therapeutically effective amount of a copper chelator that includes TTM, in combination with one or more of DEC and ATX.

The present inventors have discovered and reasoned that delivering TTM in a tablet or capsule where the TTM is protected from the stomach acids prevents the premature destruction of the TTM, such destruction creating the release of H₂S gas, thereby preventing the side effect of “sulfur burp,” and increasing the bioavailability of the TTM. Sulphur burp is a known side effect that patients taking TTM experience and is not enjoyable to the patient.

The present inventors have discovered and reasoned that preventing the DEC from releasing in the stomach by an enteric coating will prevent a known DEC side effect of nausea.

The present inventors have also discovered and reasoned that combining LEAPS technology with TTM, or with TTM and DEC, or with other co-drugs, such as ATX, or active agents selected from inhibitors of the 5-lipoxygenase enzyme (5-LO), such as DEC or zileuton, inhibitors of the LTA4 hydrolase, inhibitors of LT receptors, SFN, tert-butylhydroquinone, Bestatin, or Montelukast, in concert can have a formidable anti-cancer effect, and/or formidable anti-PAH effect.

The present inventors have also discovered and reasoned that the treatment of cancer and the treatment of PAH is further enhanced by TTM administered in an oral formulation whereby the TTM is not exposed to stomach acids. In embodiments, the oral formulation is provided with an enteric coating to release after the tablet or capsule passes the stomach as an immediate release or as an extended release form. In some embodiments, TTM is delivered as a long-acting extended-release form that provides a uniform continuous dose. In various embodiments, TTM is administered in a long-acting extended-release form that provides uniform continuous dose along with DEC, or a TTM long-acting extended-release form along with DEC and ATX, and in various embodiments, with or without co-drugs such as LEAPS peptide heteroconjugates, tart-butylhydroquinone, and SFN-based compounds. The present disclosure is also directed to pharmaceutical compositions that contain a copper chelator including TTM in a delayed release oral form that releases the copper chelator in the gastrointestinal tract after the oral form passes the stomach.

The present inventors have also discovered and reasoned that the treatment of cancer and the treatment of PAH is further enhanced by the combination of TTM and at least one active agent selected from ATX, inhibitors of the 5-lipoxygenase enzyme (5-LO), such as DEC or zileuton, inhibitors of the LTA4 hydrolase, inhibitors of LT receptors, SFN, Bestatin, tert-butylhydroquinone, or Montelukast.

According to embodiments of the present disclosure for cancer therapy or PAH therapy, TTM is provided in an enteric capsule or tablet and is not a long-acting extended-release form. According to other embodiments, TTM is provided as an extended-release from containing TTM alone, or combined with ATX, or a 5-lipoxygenase inhibitor such as DEC or Zileuton, or inhibitors of the enzyme leukotriene A4 (LTA4)-hydrolase such as Bestatin, or inhibitors of the leukotriene C4 receptor such as Montelukast, or inhibitors of leukotriene B4 receptors (BLT1, BLT2) such as LY293111, BAY-u9773, or with SFN, LEAPS peptide heteroconjugates, tart-butylhydroquinone, and/or MK, as treatment or pre-treatment. Diethylcarbamazine, Zileuton, the inhibitors of the leukotriene producing enzymes, and the leukotriene receptor inhibitors mentioned above provide multiple benefits that include inhibition of the synthesis of the leukotrienes LTB4 and LTC4 which are mediators of inflammation, and inhibition of the synthesis of the highly chemotactic LTB4, addressing inhibition of chemotaxis of inflammatory cells.

According to embodiments of the present disclosure, in addition to the foregoing drug combinations, ATX adds to the inhibition of proteases and is a powerful antioxidant that protects against mitochondrial damage. ATX adds a mechanism of action explained by upregulation of Nrf2 resulting in a normalization of the tissue and cell oxidant/anti-oxidant balance, along with inhibition of protease-induced cell damage and DNA damage-not provided by the actions of TTM.

According to embodiments of the present disclosure, in addition to the foregoing drug combinations, SFN is added to downregulate the Wnt/β-catenin self-renewal pathway in cancer, breast and lung cancer, for instance.

According to embodiments of the present disclosure, MK is added to the foregoing drug combinations for cancer treatment and pretreatment.

According to embodiments of the present disclosure, also provided is the combination of two drugs with different mechanisms of action, such as a 5-lipoxygenase inhibitor (DEC or Zileuton), or a LTA4 hydrolase inhibitor, or leukotriene receptor blockers, along with TTM as primary drivers to prevent or inhibit cancer progression, where the TTM is in a slow release format or a non-slow release format having an enteric coating to protect the TTM from the stomach's acid.

According to embodiments of the present disclosure, the copper chelator comprising TTM or a salt thereof and at least one active agent are administered separately. In an embodiment, the copper chelator is administered orally and the at least one active agent is administered intravenously, intramuscularly, depot, sublingual, inhaled, or orally.

According to embodiments of the present disclosure, also provided is a composition containing an effective amount of a copper chelator containing TTM or a salt thereof and at least one active agent selected from inhibitors of the 5-lipoxygenase enzyme (5-LO), such as DEC and zileuton, and pharmaceutically acceptable carriers and/or excipients. In various embodiments, such compositions are in an intravenous form, or in an oral form, such as a tablet, a microtablet, or a capsule. In some embodiments, the oral form provides a delayed release of the TTM in the gastrointestinal tract after passage through the stomach.

According to embodiments of the present disclosure, also provided is a composition containing an effective amount of a copper chelator containing TTM or a salt thereof, ATX, and at least one active agent selected from inhibitors of the 5-lipoxygenase enzyme (5-LO), such as DEC and zileuton, and pharmaceutically acceptable carriers and/or excipients. In various embodiments, such compositions are in an intravenous form, or in an oral form, such as a tablet, a microtablet, or a capsule. In some embodiments, the oral form provides a delayed release of the TTM in the gastrointestinal tract after passage through the stomach.

Other features and advantages of the present disclosure will be apparent from the following description of the drawings and detailed description, which should not be construed as limiting the disclosure to the examples and embodiments shown and described.

### BRIEF DESCRIPTION OF THE DRAWINGS

FIG. 1A and FIG. 1B are diagrams illustrating an oral formulation of a capsule within a capsule design according to the present disclosure. In FIG. 1A, the inner capsule is isolated from the outer capsule, according to an embodiment of the disclosure. In FIG. 1B, the inner capsule is in contact with the outer capsule.

FIG. 2 is a diagram illustrating an oral formulation of TTM, according to an embodiment of the disclosure.

FIG. 3 is a diagram illustrating an oral formulation of TTM and ATX and/or DEC, according to an embodiment of the disclosure.

FIG. 4 is a diagram illustrating an oral formulation of TTM and ATX and/or DEC, according to another embodiment of the disclosure.

FIG. 5 is a diagram illustrating an oral formulation of TTM and DEC or other co-drug, and ATX, according to an embodiment of the disclosure.

FIG. 6 is a diagram illustrating an oral formulation of TTM and DEC or other co-drug, and ATX, according to another embodiment of the disclosure.

FIG. 7 is a diagram illustrating an oral formulation of TTM and DEC or other co-drug, according to an embodiment of the disclosure.

FIG. 8 is a diagram illustrating an oral formulation of TTM and DEC, and/or ATX, and/or other co-drug, according to an embodiment of the disclosure.

FIG. 9 is a diagram illustrating an oral formulation of TTM and DEC, and/or ATX, and/or other co-drug, according to an embodiment of the disclosure.

FIG. 10 is a diagram illustrating an oral formulation that includes a multi-layer tablet of TTM, DEC, and ATX, or another co-drug, according to an embodiment of the disclosure.

FIG. 11 is a diagram illustrating an oral formulation that includes a multi-layer tablet of TTM, DEC, and ATX, or another co-drug, according to another embodiment of the disclosure.

FIG. 12 is a diagram illustrating an oral formulation that includes a multi-unit system (MUPS) providing a tablet of TTM, DEC, and ATX, or another co-drug, according to an embodiment of the disclosure.

### DETAILED DESCRIPTION

Aspects of the present disclosure are provided in the following detailed description directed to specific embodiments. Those skilled in the art will recognize that alternate embodiments may be devised without departing from the spirit or the scope of the disclosure. Additionally, well-known elements of embodiments of the disclosure will not necessarily be described in detail or will be omitted so as not to obscure the relevant details of the disclosure.

The embodiments described herein are not limiting. The described embodiments herein are not necessarily to be construed as preferred or advantageous over other embodiments. Moreover, the terms “embodiments of the disclosure”, “embodiments” or “disclosure” do not require that all embodiments of the disclosure include the discussed feature, advantage, or mode of operation.

Copper, due to its Fenton Chemistry, serves as an important cofactor for numerous proteins and enzymes involved in both physiologic and pathological process. The proteins are secreted, intracellular, or transmembraneous. There are more than fifty copper-binding proteins in the various compartments of a cell (membrane, cytoplasm, nucleus, and mitochondria) that function as copper transporters, chaperones, and enzymes. In theory, all these copper-binding proteins may be affected to various degrees by a copper chelator, such as TTM.

According to various embodiments, the copper chelator is TTM or a salt thereof, which is a highly effective copper-chelator for the purpose of the present disclosure. The terms “tetrathiomolybdate” or “TTM” as used herein refers to a MoS₄ compound, or a (MoS₄)²⁻ anion, or acid forms or salt forms thereof. As used herein, the terms “tetrathiomolybdate” or “TTM” include ammonium tetrathiomolybdate, Bis-choline tetrathiomolybdate, and ATN 224. According to various embodiments, the salt of TTM is according to formula I:

X(MoS₄),

where X is (2Li)⁺², (2K)⁺², (2Na)⁺², Mg⁺², Ca⁺², or {[N⁺(R¹)(R²)(R³)(R⁴)][N⁺(R⁵)(R⁶) (R⁷)(R⁸)]};

R¹, R², R³, R⁵, R⁶, and R⁷ are independently H, or an optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl; and

R⁴ and R⁸ are absent or independently H, or an optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl;

wherein when R⁴ is absent, R¹ and R² together with N forms an optionally substituted 5- or 6-membered aromatic ring, wherein up to 2 carbon atoms in the ring may be replaced with a heteroatom selected from the group consisting of O, N, and S;

wherein when R⁸ is absent, R⁵ and R⁶ together with N forms an optionally substituted 5- or 6-membered aromatic ring, wherein up to 2 carbon atoms in the ring may be replaced with a heteroatom selected from the group consisting of O, NH, and S;

wherein R¹ and R², R² and R³, or R² and R⁴, together with N optionally forms an optionally substituted cyclic structure;

wherein R⁵ and R⁶, R⁶ and R⁷, or R⁷ and R⁸, together with N optionally forms an optionally substituted cyclic structure;

wherein R⁴ and R⁸ may be joined by a covalent bond;

wherein R¹, R², R³, R⁵, R⁶, and R⁷ are each independently optionally substituted with one or more of OH, oxo, alkyl, alkenyl, alkynyl, NH₂, NHR⁹, N(R⁹)₂, —C═N(OH), or OPO₃H₂, wherein R⁹ is each independently alkyl or —C(═O)(O)-alkyl;

wherein R⁴ and R⁸ are each independently optionally substituted with one or more of OH, oxo, alkyl, alkenyl, alkynyl, NH₂, NHR⁹, N(R⁹)₂, —C═N(OH), or —⁺(R¹⁰)₃, wherein R¹⁰ is each independently optionally substituted alkyl; and wherein one or more —CH₂— groups in R¹, R², R³, R⁴, R⁷ and R⁸ may be replaced with a moiety selected from the group consisting of O, NH, 5, S(O), and S(O)₂.

In an embodiment, X is [N⁺(R¹)(R²)(R³)(R⁴)][N⁺(R⁵)(R⁶)(R⁷)(R⁸)] according to formula (II):

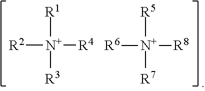

In another embodiment, where X is {[N⁺(R¹)(R²)(R³)(R⁴)][N⁺(R⁵)(R⁶)(R⁷)(R⁸)]}, [N⁺(R¹)(R²)(R³)(R⁴)] and [N⁺(R⁵)(R⁶)(R⁷)(R⁸)] are the same or different.

In another embodiment, where X is {[N⁺(R¹)(R²)(R³)(R⁴)][N⁺(R⁵)(R⁶)(R⁷)(R⁸)]}, R¹, R², R³, R⁵, R⁶, and R⁷ are independently H or C₁-C₁₀ alkyl. In another embodiment where X is {[N⁺(R¹)(R²)(R³)(R⁴)][N⁺(R⁵)(R⁶)(R⁷)(R⁸)]}, R¹, R², R³, R⁵, R⁶, and R⁷ are independently H, C₁-C₃ alkyl or C₁-C₆ alkyl. In a further embodiment where X is {[N⁺(R¹)(R²)(R³)(R⁴)][N⁺(R⁵) (R⁶)(R⁷)(R⁸)]}, R⁴ and R⁸ are independently H or C₁-C₆ alkyl.

In another embodiment, where X is {[N⁺(R¹)(R²)(R³)(R⁴)][N⁺(R⁵)(R⁶)(R⁷)(R⁸)]}, R¹, R², R³, R⁵, R⁶, and R⁷ are independently H, methyl, ethyl or propyl. In a further embodiment, where X is {[N⁺(R¹)(R²)(R³)(R⁴)][N⁺(R⁵)(R⁶)(R⁷)(R⁸)]}, each of R¹, R², R³, R⁴, R⁵, R⁶, R⁷, and R⁸ is propyl, and the compound is tetrapropylammoniumtetratinolybdate. In another embodiment, where X is {[N⁺(R¹)(R²)(R³)(R⁴)][N⁺(R⁵)(R⁶)(R⁷)(R⁸)]}, each of R¹, R², R³, R⁴, R⁵, R⁶, R⁷, and R⁸ is methyl, and the compound is tetramethylammoniumtetrathirolybdate. In a further embodiment, where X is {[N⁺(R¹)(R²)(R³)(R⁴)][N⁺(R⁵)(R⁶)(R⁷)(R⁸)]}, each of R¹, R², R³, R⁴, R⁵, R⁶, R⁷, and R⁸ is ethyl, and the compound is tetraethiylammoniumtetrathimolybdate.

In another embodiment, where X is {[N⁺(R¹)(R²)(R³)(R⁴)][N⁺(R⁵)(R⁶)(R⁷)(R⁸)]}, R¹, R², and R³ are independently H, methyl, or ethyl and R⁴ is H or an optionally substituted alkyl, alkenyl, cycloalkyl alkyl, cycloalkyl, aryl, aralkyl, heterocycloalkyl, or heteroaryl. In another embodiment, where X is {[N⁺(R¹)(R²)(R³)(R⁴)][N⁺(R⁵)(R⁶)(R⁷)(R⁸)]}, R⁵, R⁶, and R⁷ are independently H, methyl, or ethyl and R⁸ is H or an optionally substituted alkyl, alkenyl, cycloalkyl alkyl, cycloalkyl, aryl, aralkyl, heterocycloalkyl, or heteroaryl. In another embodiment, where X is {[N⁺(R¹)(R²)(R³)(R⁴)][N⁺(R⁵)(R⁶)(R⁷)(R⁸)]}, the optional substituents for R⁴ and/or R⁸ are selected from the group consisting of alkyl, OH, NH₂, and oxo. In another embodiment, where X is {[N⁺(R¹)(R²)(R³)(R⁴)][N⁺(R⁵)(R⁶)(R⁷)(R⁸)]}, one or more —CH₂— groups of R⁴ and/or R⁸ is replaced with a moiety selected from O, NH, S, S(O), and S(O)₂.

In another embodiment, where X is {[N⁺(R¹)(R²)(R³)(R⁴)][N⁺(R⁵)(R⁶)(R⁷)(R⁸)]}, R¹, R², R³, R⁵, R⁶, and R⁷ are independently methyl and R⁴ and R⁸ is each optionally substituted alkyl.

In another embodiment, where X is {[N⁺(R¹)(R²)(R³)(R⁴)][N⁺(R⁵)(R⁶)(R⁷)(R⁸)]}, each of R¹, R², R³, R⁵, R⁶, and R⁷ is independently methyl and R⁴ and R⁸ is each optionally substituted ethyl.

In another embodiment, where X is {[N⁺(R¹)(R²)(R³)(R⁴)][N⁺(R⁵)(R⁶)(R⁷)(R⁸)]}, R¹, R², R³, R⁵, R⁶, and R⁷ are independently methyl and R⁴ and R⁸ is each substituted ethyl, wherein the substituent is a hydroxyl. In another embodiment, where X is {[N⁺(R¹)(R²)(R³)(R⁴)][N⁺(R⁵)(R⁶) (R⁷)(R⁸)]}, each of R¹, R², R³, R⁵, R⁶, and R⁷ is independently methyl and R⁴ and R⁸ is each —CH₂CH₂—OH.

In another embodiment, where X is {[N⁺(R¹)(R²)(R³)(R⁴)][N⁺(R⁵)(R⁶)(R⁷)(R⁸)]}, R¹, R², R³, R⁵, R⁶, and R⁷ are independently methyl, R⁴ and R⁸ is each optionally substituted alkyl, and the compound is tetramethylammoniumtetrathimolybdate. In another embodiment, where X is {[N⁺(R¹)(R²)(R³)(R⁴)][N⁺(R⁵)(R⁶)(R⁷)(R⁸)]}, each of R¹, R², R³, R⁵, R⁶, and R⁷ is independently methyl, R⁴ and R⁸ is each optionally substituted ethyl, and the compound is tetramethylammoniumtetrathimolybdate. In a further embodiment where X is {[N⁺(R¹)(R²)(R³) (R⁴)][N⁺(R⁵)(R⁶)(R⁷)(R⁸)]}, R¹, R², R³, R⁵, R⁶, and R⁷ are independently methyl, R⁴ and R⁸ is each substituted ethyl, wherein the substituent is a hydroxyl, and the compound is tetramethylammoniumtetrathimolybdate. In another embodiment, where X is {[N⁺(R¹)(R²)(R³) (R⁴)][N⁺(R⁵)(R⁶)(R⁷)(R⁸)]}, R¹, R², R³, R⁵, R⁶, and R⁷ are independently methyl, R⁴ and R⁸ is each —CH₂CH₂—OH, and the compound is tetramethylammoniumtetrathimolybdate.

In another embodiment, the copper chelator compound is bis-choline tetrathiomolybdate.

In another embodiment, the copper chelator compound according to formula (I) is:

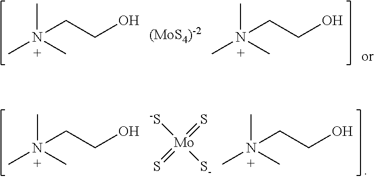

Table 1 provides non-limiting embodiments where X is {[N⁺(R¹)(R²)(R³)(R⁴)][N⁺(R⁵) (R⁶)(R⁷)(R⁸]}

TABLE 1

| | R¹ | R² | R³ | R⁴ | R⁵ | R⁶ | R⁷ | R⁸ |
|---|---|---|---|---|---|---|---|---|
| 1 | H | H | H | H | H | H | H | H |
| 2 | CH₃ | CH₃ | CH₃ | CH₃ | CH₃ | CH₃ | CH₃ | CH₃ |
| 3 | ethyl | ethyl | ethyl | ethyl | ethyl | ethyl | ethyl | ethyl |
| 4 | propyl | propyl | propyl | propyl | propyl | propyl | propyl | propyl |
| 5 | butyl | butyl | butyl | butyl | butyl | butyl | butyl | butyl |
| 6 | pentyl | pentyl | pentyl | pentyl | pentyl | pentyl | pentyl | pentyl |
| 7 | H | H | H | H | CH₃ | CH₃ | CH₃ | CH₃ |
| 8 | H | H | H | H | ethyl | ethyl | ethyl | ethyl |
| 9 | H | H | H | H | propyl | propyl | propyl | propyl |
| 10 | H | H | H | H | butyl | butyl | butyl | butyl |
| 11 | CH₃ | CH₃ | CH₃ | CH₃ | ethyl | ethyl | ethyl | ethyl |
| 12 | CH₃ | CH₃ | CH₃ | CH₃ | propyl | propyl | propyl | propyl |
| 13 | CH₃ | CH₃ | CH₃ | CH₂CH₂OH | CH₃ | CH₃ | CH₃ | CH₂CH₂OH |

In another embodiment, where X is {[N⁺(R¹)(R²)(R³)(R⁴)][N⁺(R⁵)(R⁶)(R⁷)(R⁸)]}, each of [N⁺(R¹)(R²)(R³)(R⁴)] and [N⁺(R⁵)(R⁶)(R⁷)(R⁸)] is independently:

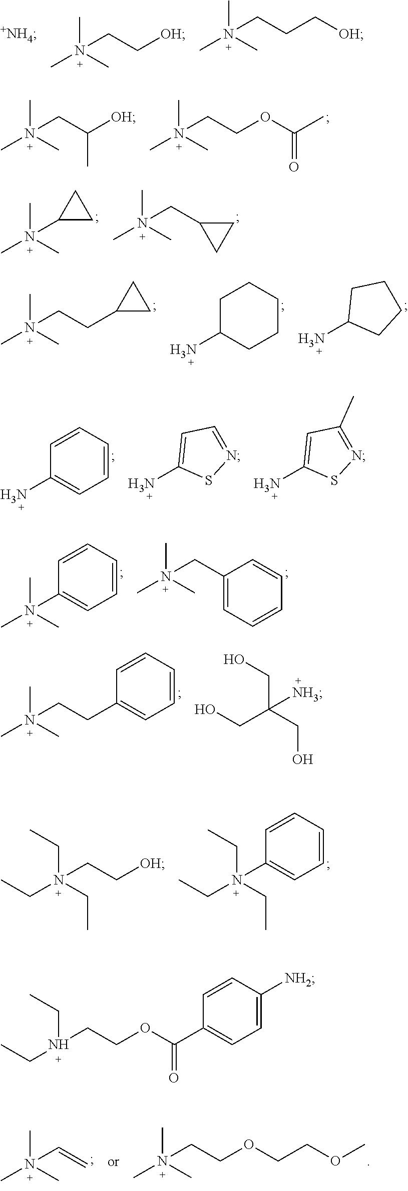

In another embodiment, where X is {[N⁺(R¹)(R²)(R³)(R⁴)][N⁺(R⁵)(R⁶)(R⁷)(R⁸]} at least one of [N⁺(R¹)(R²)(R³)(R⁴)] and [N⁺(R⁵)(R⁶)(R⁷)(R⁸)] is:

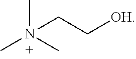

In another embodiment, where X is {[N⁺(R¹)(R²)(R³)(R⁴)][N⁺(R⁵)(R⁶)(R⁷)(R⁸)]}, both [N⁺(R¹)(R²)(R³)(R⁴)] and [N⁺(R⁵)(R⁶)(R⁷)(R⁸] are:

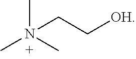

In another embodiment, where X is {[N⁺(R¹)(R²)(R³)(R⁴)][N⁺(R⁵)(R⁶)(R⁷)(R⁸)]}, R¹, R², R³, and R⁴ are each independently H or alkyl. In another embodiment, R⁵, R⁶, R⁷, and R⁸ are each independently H or alkyl.

In another embodiment, where X is {[N⁺(R¹)(R²)(R³)(R⁴)][N⁺(R⁵)(R⁶)(R⁷)(R⁸]}, R⁴ and R⁸ are joined by a covalent bond. For example, if R⁴ and R⁸ are both methyl, when R⁴ and R⁸ are joined by a covalent bond, it can form an ethylene link between the two nitrogen atoms as illustrated below:

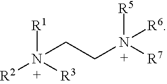

In another embodiment, where X is {[N⁺(R¹)(R²)(R³)(R⁴)][N⁺(R⁵)(R⁶)(R⁷)(R⁸)]}, R⁴ and R⁸ are both optionally substituted alkyl group joined by a covalent bond.

In another embodiment, where X is {[N⁺(R¹)(R²)(R³)(R⁴)][N⁺(R⁵)(R⁶)(R⁷)(R⁸)]}, R¹, R², R³, R⁵, R⁶, and R⁷ are independently H, methyl, ethyl or propyl and R⁴ and R⁸ are joined by a covalent bond. In another embodiment, R⁴ and R⁸ is each independently an optionally substituted alkyl group. In another embodiment, the optional substituents for R⁴ and R⁸ is N⁺(R¹⁰)³, wherein R¹⁰ is optionally substituted alkyl. In another embodiment, one or more —CH₂— groups of R⁴ and R⁸ are replaced with a moiety selected from the group consisting of O, NH, S, S(O), and S(O)₂.

In another embodiment, where X is {[N⁺(R¹)(R²)(R³)(R⁴)][N⁺(R⁵)(R⁶)(R⁷)(R⁸)]}, X is:

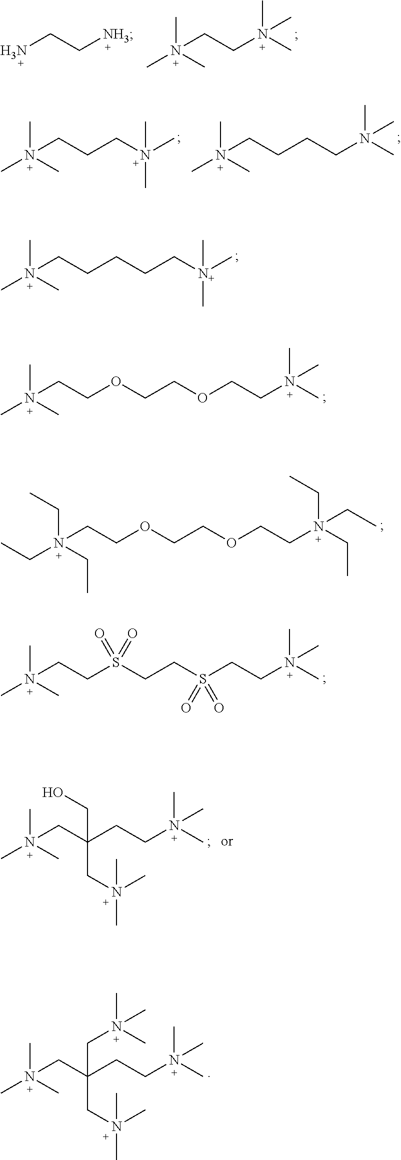

In another embodiment, where X is {[N⁺(R¹)(R²)(R³)(R⁴)][N⁺(R⁵)(R⁶)(R⁷)(R⁸)]}, R¹ and R² are each independently H, methyl, or ethyl, and R³ and R⁴ are each independently an optionally substituted alkyl, aryl, or aralkyl group. In another embodiment, where X is {[N⁺(R¹) (R²)(R³)(R⁴)][N⁺(R⁵)(R⁶)(R⁷)(R⁸)]}, R⁵ and R⁶ are each independently H, methyl, ethyl, or propyl, and R⁷ and R⁸ are each independently an optionally substituted alkyl, aryl, or aralkyl group. In another embodiment, the optional substituents for R³, R⁴, R⁷, and R⁸ are OH.

In another embodiment, where X is {[N⁺(R¹)(R²)(R³)(R⁴)][N⁺(R⁵)(R⁶)(R⁷)(R⁸]}, [N⁺(R¹)(R²)(R³)(R⁴)] and/or [N⁺(R⁵)(R⁶)(R⁷)(R⁸)] is independently:

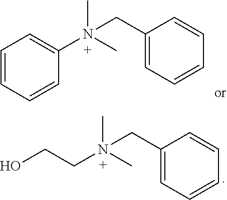

In another embodiment, where X is {[N⁺(R¹)(R²)(R³)(R⁴)][N⁺(R⁵)(R⁶)(R⁷)(R⁸)]}, R¹ and R⁴ are each independently H, methyl, ethyl, or propyl and R² and R³ together with N may form an optionally substituted cyclic structure.

In another embodiment, where X is {[N⁺(R¹)(R²)(R³)(R⁴)][N⁺(R⁵)(R⁶)(R⁷)(R⁸)]}, R⁵ and R⁸ are each independently H, methyl, ethyl, or propyl, and R⁶ and R⁷ together with N may form an optionally substituted cyclic structure. In another embodiment, one or more —CH₂— groups in R², R³, R⁶, and R⁷ is replaced with a moiety selected from the group consisting of O, NH, S, S(O), and S(O)₂.

In another embodiment, where X is {[N⁺(R¹)(R²)(R³)(R⁴)][N⁺(R⁵)(R⁶)(R⁷)(R⁸]}, [N⁺(R¹)(R²)(R³)(R⁴)] and/or [N⁺(R⁵)(R⁶)(R⁷)(R⁸)] is independently:

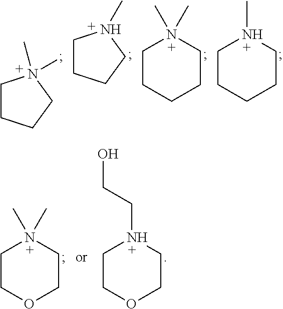

In another embodiment, where X is {[N⁺(R¹)(R²)(R³)(R⁴)][N⁺(R⁵)(R⁶)(R⁷)(R⁸)]}, R⁴ and/or R⁸ is absent and R¹ and R² and/or R⁵ and R⁶ together with N form an optionally substituted 5- or 6-membered aromatic ring, wherein up to 2 carbon atoms in the ring may be replaced with a heteroatom selected from the group consisting of O, N, and S.

In another embodiment, where X is {[N⁺(R¹)(R²)(R³)(R⁴)][N⁺(R⁵)(R⁶)(R⁷)(R⁸)]}, [N⁺(R¹)(R²)(R³)(R⁴)] and/or [N⁺(R⁵)(R⁶)(R⁷)(R⁸)]} is independently:

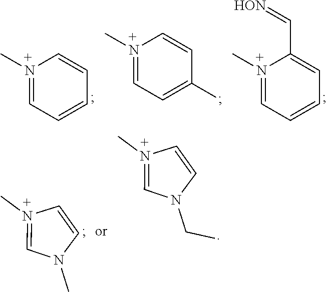

In another embodiment, where X is {[N⁺(R¹)(R²)(R³)(R⁴)][N⁺(R⁵)(R⁶)(R⁷)(R⁸)]}, R¹, R², R³, R⁴, R⁵, R⁶, R⁷, and R⁸ are each H.

In another embodiment, the copper chelator compound is ammonium tetrathiomolybdate (NH₄)₂MoS₄ (ATTM). In embodiments, ATTM is combined with other copper chelator compounds, such as ammonium trithiomolybdate (NH₄)₂MoOS₃.

In another embodiment, the copper chelator compound is Bis-choline tetrathiomolybdate C₁₀H₃₀MoN₂O₂S₄ (sometimes referred to as ATN 224) that substitutes for the ATTM, or is combined with the ATTM, or is combined with other copper chelator compounds, such as ammonium trithiomolybdate (NH₄)₂MoOS₃.

In further embodiments, cancer and/or PAH in a patient is treated by administering a therapeutically effective amount of TTM or a salt thereof. In some embodiments, the copper chelator includes ATTM, and in some embodiments the copper chelator further includes ammonium trithiomolybdate (NH₄)₂MoOS₃. In various embodiments, the amount of TTM delivered to the patient is individualized. In an embodiment, the therapeutically effective amount of the copper chelator delivered to the patient is between 20 mg and 300 mg of TTM/day. In various embodiments, the amount of TTM is adjusted according to the level of ceruloplasmin in the plasma. An effective copper chelation is achieved when the plasma ceruloplasmin level approaches about 50% of the normal level; i.e. about 15-17 mg/dl.

“Administering” has its customary and ordinary meaning as understood by one of skill in the art in view of this disclosure. It refers to providing a pharmaceutical agent or composition to a subject, and includes, but is not limited to, administering by a medical professional and self-administration.

An “effective amount” of a drug, compound, or composition containing such compound, refers to the amount sufficient to achieve a desired biological and/or pharmacological effect according to a selected administration form, route, and/or schedule. The phrases “effective amount” and “therapeutically effective amount” are used interchangeably. Those of ordinary skill in the art will further understand that an “effective amount” may be administered to a subject in a single dose, or through use of multiple doses, in various embodiments.

“The terms “subject” or “patient” as used herein are used interchangeably and mean all members of the animal kingdom (e.g. humans).

According to various embodiments, dosing of TTM for treating cancer, for cancer adjuvant therapy, and/or for treating PAH is as follows:

if the ceruloplasmin level is between 15 mg/dL and 18 mg/dL, then the TTM dose will be stable at 60 mg TTM given three times a day for a total of 180 mg per day;

if the ceruloplasmin level falls below 15 mg/dL, then the TTM dose will be a total of 120 mg per day and further reduced if needed;

if the ceruloplasmin level is above 20 mg/dL, then the TTM dose will be a total of 240 mg per day, and up to 300 mg per day if needed.

According to various embodiments, the copper chelator is administered in a composition containing pharmaceutically acceptable carriers and/or excipients. In embodiments, the composition is administered in an oral form, such as a tablet, a microtablet, a capsule, or a sachet. In some embodiments, the copper chelator is in a composition as an oral form with specific carriers, matrix compounds, and/or excipients that provide a delayed release of the copper chelator in the gastrointestinal tract after passage through the stomach. In general, the carriers, matrix, and/or other excipients are selected to facilitate extended and controlled release of the copper chelator, enabling optimal intestinal uptake and absorption, guarantee stability for storage, and minimizing risk of alcohol-related dose dumping. Moreover, the carriers, matrix, and/or excipients are selected such that destruction by gastric acid is removed or avoided. For this objective, suitable coating materials for enteric coating are applied. For example, various embodiments of the oral forms of the composition include an enteric coating of the tablet, micro-tablet, capsule, or individual pellets and beads in the capsule. Also, in some embodiments, multiple coating layers are applied, e.g., a coating layer for enteric coating and an extended-release coating layer. In some embodiments, a layer is added to avoid burst release. Also, in various embodiments, different dosage forms are combined, such as capsules in capsules, or mini-tablets in capsules. In some embodiments, capsules or mini-tablets are combined with a filler and an adsorbent which can be an antacid, between the TTM and the enteric coating to minimize exposure to liquid water and stomach acid as enteric coating do not seal and the TTM degrades quickly when exposed to any stomach acid that can seep through the enteric coating. By the absorbent including an antacid compound, any acid that seeps through the enteric coating will be neutralized and protect the TTM from degradation.

The term “pharmaceutically acceptable” as used herein refers to drugs, compounds, compositions, and dosage forms which are suitable for use in human beings and animals and without excessive toxicity, irritation, allergic response, or any other problem or complication.

The term “oral dose,” “oral dosage,” or “oral formulation” as used herein means a dosage form that is administered by mouth, for absorption through the mucous membranes of the mouth and/or, after swallowing, through the gastrointestinal tract. Such oral dosage forms include but are not limited to solutions, syrups, suspensions, emulsions, gels, powders, granules, capsules, tablets, buccal dosage forms and sublingual dosage forms.

As used herein, “enteric coating” has its customary and ordinary meaning as understood by one of skill in the art in view of this disclosure. An enteric coating is a barrier applied to oral medication that prevents dissolution or disintegration in the gastric environment. This protects drugs from the acidity of the stomach and helps to release the drug in a desired portion of the gastrointestinal tract, for example the upper tract of the intestine. Many enteric coatings work by presenting a surface that is stable at the intensely acidic pH found in the stomach, but breaks down rapidly at a higher pH (alkaline pH). For example, they will not dissolve in the gastric acids of the stomach (pH ˜3), but they will in the alkaline (pH 7-9) environment present in the small intestine.

In embodiments, the filler or absorbent is an antacid, and the antacid is one or more of aluminum hydroxide, calcium carbonate, magnesium carbonate, magnesium hydroxide, magnesium trisilicate, sodium bicarbonate, alginate, or a combination thereof.

According to various embodiments, the oral coating layer is an extended-release coating, whereby the extended-release coating creates a stable administration of the TTM, whereby it is released over a period of time, creating a long- or extended-release of TTM and reducing or eliminating the need for taking several dosages over a given time period. Such extended release coatings are based on different natural or man-made polymers, such as, but not limited to, ethyl celluloses, hydroxypropylmethylcelluloses, methylcelluloses, hydroxypropylcelluloses, hydroxyethylcelluloses, and sodium carboxy-methylcellulose.

The term “extended-release” as used herein refers to pharmaceutical compositions that are designed to slowly release an active ingredient, medication, or drug in the body over an extended period of time. An extended-release formulation of a pharmaceutical composition helps maintain a consistent level of the active ingredient, medication, or drug in the body. It also allows for reduced dosing frequency, increases compliance as the patient has less pills a day to consume, and helps lower the risk of side effects.

Embodiments of enteric coatings and extended release coatings layers are external and/or internal to the tablets, beads, or pellets and in some embodiments are combined to protect from acidic stomach content and/or to create a controlled release profile over an extended amount of time.

According to various embodiments, an extended release formulation is designed as matrix tablets or pellets in a capsule where either a hydrophilic, hydrophobic, or lipophilic polymer is used as carrier or a lipid matrix

In embodiments of the hydrophilic matrix system, TTM is dispersed throughout a polymer matrix of hydrophilic material. The rate of drug release is controlled by both diffusion and erosion. When water is absorbed by the matrix, the matrix swells, and the polymer on the surface of the tablet hydrates. TTM dissolves and is released by a combination of diffusion out of the matrix, through the gel layer, and as a result of the erosion of the matrix itself. In embodiments, formulations of the matrix system are enterically coated to inhibit destruction of the copper chelator by stomach acid. Alternatively, the matrix based system is put in an enteric capsule, where the capsule is either made from an enteric material, or is coated and sealed enterically and includes a barrier of antacid to prevent acid that seeps through the enteric coating for interacting with the TTM.

In embodiments of the hydrophobic matrix system, TTM is dispersed throughout a polymer matrix of inert hydrophobic material, e.g., polymer or lipid. In this embodiment, the hydrophobic matrix undergoes no or minimal swelling on contact with water. When water enters the matrix, TTM dissolves and is predominately released by diffusion out of the matrix. In embodiments, formulations of the matrix system are enterically coated to inhibit destruction of the copper chelator by stomach acid. Alternatively, the matrix based system is put in an enteric capsule, where the capsule is either made from an enteric material, or is coated and sealed enterically. As enteric coatings not seal, the TTM is isolated from the inside of an enteric coating to avoid the stomach acid that initially seeps through the enteric coating from contacting the TTM.

In another embodiment, suitable polymers, lipid carriers, or a combination of both, are used for a hot-melt extrusion process to make an extended-release material that can further be processed into tablets, micro-tablets, pellets, or beads. In embodiments, a coating layer(s) for enteric coating and/or controlled release adds enteric protection and helps avoid burst release. In some embodiments, another barrier is coated onto the delivery form, either on top or below the enteric coating. This barrier layer is designed such as to improve resistance to diffusion of moisture through the enteric film, i.e., from reaching the copper chelator as the enteric coating starts to dissolve. The barrier agent layer is such that it either absorbs water, neutralizes the acid that seeps through the enteric coating, prevents the TTM from being in contact with the acid, or slowly dissolves (e.g., a functional controlled-release polymer), or is a lipophilic (hydrophobic) coating substance, e.g., lipids and other hydrophobic excipients, or provides antacid properties.

According to various embodiments, the TTM is packaged in an extended-release system that includes a reservoir system, whereby a core containing TTM is surrounded by an insoluble polymer membrane of suitable extended release polymers. In embodiments, the TTM is contained in a single core, but some embodiments also include additional subunits, such as beads, pellets, or mini-tablets, containing the drug. Some embodiments include a coating layer to add enteric protection and to help avoid burst release.

In embodiments, TTM is packaged in a capsule with multiple different subunits, such as microtablets, pellets, or beads, with different release characteristics allowing for a multimodal IR (immediate release) plus extended release (ER). In embodiments, the capsule is enterically coated or the subunits are enterically coated. In embodiments, an additional moisture-diffusion-limiting barrier layer minimizes acid-related destruction of the copper chelator from the stomach acid that would otherwise leak through the enteric barrier.

According to various embodiments the TTM or copper chelator is packaged in an extended-release system using an osmotic-release system that includes a drug-containing core surrounded by an insoluble but semipermeable membrane capsule or coating. This membrane contains an orifice through which the soluble drug is forced by osmotic pressure that builds up inside the capsule on contact with water. In various embodiments, the semipermeable membrane is made of polymeric materials, such as, but not limited to, cellulose acetate polymers, cellulose esters, cellulose ethers, agar acetates, amylose triacetates, betaglucan acetates, poly(vinylmethyl)ether copolymers, poly(orthoesters), polyacetals and selectively permeable poly(glycolic acid), poly(lactic acid) derivatives, as well as Eudragits. In embodiments, this delivery system is enterically coated to ensure that no copper chelator is released in the stomach and that the stomach acid cannot leak through the enteric coating and contact the TTM.

Some embodiments of the manufacturing of the extended-release systems disclosed herein include a need to prevent oxidation of TTM in the manufacturing process, and embodiments of methods to prevent this oxidation include processing in an inert gas or in an oil or solvent or other non-oxidative material that prevents the TTM from being exposed to oxygen. In embodiments, the inert gas is Argon or Nitrogen. Also in some embodiments, processes with a short residence time in the system are used to minimize exposure of TTM to air where the product is immediately collected and stored under inert gas. Embodiments of the manufacturing process are water free and do not expose TTM to water and hydrolysis. Thus, for these manufacturing processes, solvents other than water are used.

In another embodiment of the manufacturing process, direct compaction is used to manufacture the tablets. Whereby in this process a mixture of TTM and suitable excipients are fed directly or individually to a tablet press, using standard extended-release excipients (such as a HPMC, PEO or Eudragit RL, RS, cross-linked PVA) and/or more advanced excipients including polymer mixtures. In some embodiments, other excipients for lubrication, stabilization, coloring, taste-masking, or for use as fillers and binders are added in the powder mixture. Such excipients include, but are not limited to, metal soaps such as magnesium stearate, sodium stearyl fumarate, croscarmellose sodium, modified starches, modified lactoses, dextrins, glucose, sucrose, sorbitol dicalcium phosphates, vitamins, colorants, sugar alcohols, crospovidone, polymers and copolymers, silica compounds, silicone or alginates, microcrystalline cellulose, hydroxypropylcellulose. Good flowability of the powder and low tendency for segregation of the powder mixture are desired. Embodiments of the process are carried out in batch or in continuous mode.

In another embodiment, roller compaction followed by milling and screening is applied to make granules which are then mixed with lubricants and other excipients for tableting. Standard extended-release excipients are used (such as a HPMC, PEO or Eudragit RL, RS, cross-linked PVA) or more advanced excipients including polymer mixtures. In some embodiments, other excipients for stabilization, coloring, taste-masking, or for use as fillers and binders added in the powder mixture. Embodiments of the process are carried out in batch or in continuous mode.

In another embodiment, wet granulation, e.g., via massing and screening or high-shear wet granulation or twin-screw wet granulation, with solvents that do not destroy TTM are used to make granules, followed by a drying process (e.g., via tray drying, fluid bed drying, conveyer belt drying and other methods) to produce dry granules which are used for tableting. In some embodiments, the solvents used do not contain water. Standard extended-release excipients are used (such as a HPMC, PEO or Eudragit RL, RS, cross-linked PVA) or more advanced excipients including polymer mixtures. In some embodiments, other excipients for stabilization, coloring, taste-masking, or for use as fillers and binders are added in the powder mixture. Embodiments of the process are carried out in batch or in continuous mode. In various embodiments, granules are filled in capsules and sachets, or are used for tableting if mixed with an external phase, such as lubricants or binders.

In another embodiment, granules are produced via wet granulation in extruders where a suitable solvent is added and matrix materials and TTM are added either separately or as a premix.

Extruders include twin-screw extruders, radial screw extruders, roll extruders or Koller press extruders. Subsequently to extrusion, granules are dried via methods as described herein. Materials are selected based on required formulation and biopharmaceutical requirements as described herein. In various embodiments granules are filled in capsules and sachets, or are used for tableting if mixed with an external phase, such as lubricants or binders.

In another embodiment, hot-melt extrusion is applied where powders or powder mixtures containing TTM, a matrix material for extended release, and other excipients are fed to a hot-melt extruder. The extruded strand is cooled and milled, or is directly processed into pellets and beads or to tablets via calandering. In embodiments, milled material is mixed with suitable excipients and is tableted on a tableting machine. Embodiments of the process are carried out in batch or in continuous mode.

In yet another embodiment, additive manufacturing technology, also known as 3D printing, is applied to make tablets of a desired release profile. For some embodiment of such a process, filaments are manufactured that contain the TTM and delayed-release matrix materials as described herein. These filaments are made by extrusions process as described herein using different types of extruders including single-screw, double-screw or ram extruders. Filaments are then used to print tablets via thermal technique, such as fused deposition modeling. Alternatively, melt from extruders are directly used to cast tablets via additive manufacturing technology. In embodiments, other additive manufacturing technologies are applied such as powder bed printing, inject printing, VAT polymerization, direct-wise printing, and others. Materials for printing include delayed-release artificial and natural polymers, starches, lactoses, hydroalcohols, lipids, and other natural products.

For coating the tablets, in various embodiments, standard or advanced drum coaters are used that also can be used to carry out multiple coating steps. For coating of micro-tablets, pellets, and beads, in various embodiments, fluidized bed coaters are used. Sprayed solutions or suspensions contain the required polymers for enteric coating, or extended release and suitable colors, pigments, surfactants, plasticizers, and other components. Alternatively, in embodiments, spray congealing or dip coating is applied.

According to various embodiments, the extended-release system contains TTM from a minimum of 20 mg to up to 300 mg with an approximate release period of up to about 24-hours.

According to various embodiments, the extended release tablet further contains DEC as a co-drug. Embodiments of suitable formulations that minimize chemical interaction between DEC and TTM are disclosed.

According to various embodiments, the extended-release tablet further contains AXT as a co-drug. Embodiments of suitable formulations that minimize chemical interaction between AXT and TTM are disclosed.

According to various embodiments, the extended-release tablet contains TTM, ATX, and DEC and/or another co-drug.

Various embodiments of the present disclosure for cancer and/or PAH therapy combine TTM in an extended release tablet design of TTM alone or combined as a multi drug form with one or more co-drugs such as 5-lipoxygenase inhibitors, such as DEC or Zileuton, or inhibitors of the enzyme leukotriene A4 [LTA4]-hydrolase such as Bestatin, or inhibitors of the leukotriene C4 receptor such as Montelukast, or inhibitors of leukotriene B4 receptors (BLT1, BLT2) such as LY293111, BAY-u9773, or the inclusion of SFN, or AXT or MK, or LEAPS peptide heteroconjugates, with tart-butylhydroquinone, as pre-treatment. Both DEC or Zileuton or the other inhibitors of the leukotriene producing enzymes or leukotriene receptor inhibitors mentioned above provide multiple benefits that include inhibition of the synthesis of the leukotrienes LTB4 and LTC4 which are mediators of inflammation, and inhibition of the synthesis of the highly chemotactic LTB4 addressing inhibition of chemotaxis of inflammatory cells.

In various embodiments, the copper chelator compound is ammonium tetrathiomolybdate [NH₄]2MoS₄ (ATTM). In some embodiments, ATTM is combined with other copper chelator compounds, such as ammonium trithiomolybdate [NH₄]2MoOS₃ or ATN 224.

According to various embodiments, cancer and/or PAH in a patient is treated by administering a therapeutically effective amount of a copper chelator TTM, ATTM, or ATN 224, or a salt thereof. In an embodiment, the copper chelator includes ATTM, and in some embodiments the copper chelator further includes ammonium trithiomolybdate [NH₄]2MoOS₃. The amount of TTM delivered is individualized. In an various embodiments, the therapeutically effective amount of the copper chelator delivered to the patient is between 20 mg and 300 mg of TTM/day. In embodiments, the amount of TTM is adjusted according to the level of the ceruloplasmin in plasma. An effective copper chelation is achieved when the plasma ceruloplasmin level approaches 50% of the normal level; i.e., about 15-17 mg/dl.

According to various embodiments, the copper chelator is administered in a composition containing pharmaceutically acceptable carriers and/or excipients. The composition is administered in an oral form, such as a tablet, a microtablet, a capsule filled with pellets or powder, or a sachet. In some embodiments, the copper chelator is in composition of an oral form with specific carriers, matrix compounds, and/or excipients that provide a delayed release of the copper chelator in the gastrointestinal tract after passage through the stomach. Such matrix materials can be, but are not limited to, hydroxypropyl methylcellulose (HMPC), gums, alginates, lipids of various compositions, polyvinyl acetates, polyvinylpyrrolidones, methacrylate copolymers, polyethylene glycols (PEG)/polyethylene oxides (PEO), and others. In general, the carriers, matrix, and/or other excipients are selected to facilitate extended (sustained) and controlled release of the copper chelator, enabling optimal intestinal uptake and absorption, to guarantee stability for storage, and possibly minimizing risk of alcohol-related dose dumping. Moreover, the carriers, matrix, and/or excipients are selected such that destruction by gastric acid is avoided. For this objective, suitable coating materials for enteric coating are applied. In embodiments, such a coating is applied externally on the tablet or on drug-containing pellets individually. For example, embodiments of oral forms of the composition include an enteric coating of the tablet, micro-tablet, capsule, or of individual pellets and beads in the capsule. Also, in embodiments, multiple coating layers are applied, e.g., a coating layer for enteric coating and an extended release coating layer. Also, in embodiments, a layer is added to avoid burst release and prevent acid that leaks through the enteric costing from interacting with the TTM, such layers provide antiacid characteristics. Enteric coatings contain typically pH-sensitive polymers or particles, such as, but not limited to, cellulose acetate phthalate, cellulose acetate trimellitate, shellacs, polyvinyl acetate phthalate, hydroxy-propylmethylcellulose phthalate, hydroxypropylmethylcellulose acetate succinate, poly-methacrylic acids, poly-ethyl acrylates, or poly-methacrylates at various mixtures, amylose starches and other starches, dextrins, plant proteins (e.g., zein and others), fatty acids, lipids including modified lipids, waxes and other.

In another embodiment, suitable polymers or lipid carriers or a combination of both are used for a hot-melt extrusion process to make the extended-release material which can further be processed into tablets, micro tablets, or pellets and beads. In embodiments, coating layer(s) for enteric coating and controlled release coating add enteric protection and help avoid burst release. In some embodiments, another barrier is coated onto the delivery form, either on top or below the enteric coating. This barrier layer is designed such as to improve resistance to diffusion of moisture through the enteric film, i.e., from reaching the copper chelator as the enteric coating starts to dissolve. The barrier agent layer is such that it either absorbs water, neutralizes the acid that seeps through the enteric coating or slowly dissolves (e.g., a functional controlled-release polymer) or may be a lipophilic (hydrophobic) coating substance, e.g., lipids and other hydrophobic excipients.

According to various embodiments, the extended-release system contains TTM from a minimum of 20 mg to up to 300 mg with a TTM in an approximate release period of about 24-hours.

According to various embodiments, the extended release system also contains DEC or ATX as co-drugs. In various embodiments, suitable formulations that minimize chemical interaction between DEC and TTM and ATX are designed. In some embodiments, this means to create mixtures of the drug and co-drugs and to investigate chemical stability and to choose formulations based on the outcome. Should a chemical interaction occur, embodiments of the formulation techniques will separate the two chemicals spatially. In some embodiments, this is achieved by making multi-layer tablets with layer 1 containing TTM, and a layer 2 and a layer 3 containing a co-drug. In other embodiments, pellets of two types are made with the methods described herein, with pellet (or bead) type 1 containing TTM, and pellet (or bead) type 2 containing the co-drug. Both pellet (or bead) types can be processed in different ways, e.g., by compressing them into one tablet, or coating them separately and embedding them in a matrix material, called multi-unit pellet system (MUPS). In embodiments, the produced tablets are coated enterically or with extended release coating. In embodiments, the matrix material of the MUPS is made of extended release matrix materials as described herein. Another embodiment is to prepare multilayer coating of an inert bead, having different APIs in different coating layers. Thereafter, the beads are filled in capsules with release modifiers. In another embodiment, TTM loaded particles (pellets) are prepared as per the methods described herein and thereafter are coated with polymers/excipients having other API. Thereafter, the coated particles are delivered as tablets with excipients or filled in capsules with release modifiers and excipients. Another embodiment is to use an active coating of the co-drug. In this case, TTM is contained in the core of the tablet and a suspension or solution containing the co-drug is sprayed on the tablets via a conventional coating process. The tablet can still be enterically coated or coated with an extended release polymer.

Embodiments of the present disclosure include methods for cancer and/or PAH treatment in a patient that includes administering TTM alone, or at least one other active agent, or co-drug, in combination with TTM.

Copper is highly angiogenic, and TTM enhanced by DEC is anti-angiogenic. The combination of TTM with ATX, or TTM, DEC, and ATX provides a more effective inhibition of NF-kappaB activation and therefore inhibition of production of inflammatory mediators. While DEC inhibits the synthesis of leukotrienes, AXT, via inhibition of MAPK, inhibits the action of leukotrienes. Moreover, by using the combination of TTM and DEC, or TTM, DEC, and ATX, the present inventors are employing strategies that (1) inhibit chemotaxis of inflammatory cells, (2) decrease vascular permeability and leak, (3) decrease the activity of the master inflammatory mediator transcription factor NF-kappaB, and (4) decrease VEGF production and action, vital to combating cancer. The summary mechanisms of action for DEC are inhibition of the enzyme 5-lipoxygenase, inhibition of oxidants, and inhibition of NF-kappaB-dependent gene transcription. In the aggregate, by such molecular mechanisms, DEC inhibits chemotaxis and preserves normal endothelial cell function, i.e., it also decreases vascular leak. Note that vascular permeability (vascular leak), plays a role in PAH and cancer and it is part of the inflammatory process. To summarize the mechanisms of actions of ATX: this non-toxic and bioavailable carotene acts as a powerful antioxidant via upregulation of the expression of Nrf2, it is an antiprotease, it stabilizes mitochondrial metabolism, protects against DNA damage, it is anti-inflammatory and anti-fibrotic, protects endothelial cells against damage and it has properties shown to inhibit cancer cell growth,

A supporting reason for using TTM to curtail cancer and/or PAH includes the fact that the molybdenum in TTM binds with a very high affinity to the copper atoms in the catalytic centers of at least 54 copper-containing proteins, many of which are involved in angiogenesis and cell growth. This particular affinity of molybdenum is independent of the copper chelation property of TTM. The combination of two drugs with different mechanisms of action (a 5-lipoxygenase inhibitor like DEC or Zileuton) plus TTM are primary drivers to fight cancer and/or PAH. ATX adds mechanisms of action explained by upregulation of Nrf2 resulting in a normalization of the tissue and cell oxidant/anti-oxidant balance, inhibition of protease-induced cell damage and DNA damage-not provided by the actions of TTM.

A supporting reason for using TTM and DEC or other co-drugs, such as ATX and others disclosed herein to disrupt cancer and/or PAH includes the fact that TTM decreases the activity of two very important transcription factors: HIF-1-alpha, which is needed for the production of the highly angiogenic VEGF and plays a role in cancer, and the transcription factor NF-kappaB, which is a switchboard for the production of a very large number of inflammatory mediators. This dampening of transcription factors is therapeutically important because it will reduce angiogenic cell growth and inflammation found in human patients.

TTM can also induce apoptotic cell death of apoptosis-resistant abnormal vascular cells—another favorable anti-cancer and anti-PAH treatment effect.

A supporting reason for using TTM to assist in combating cancer and/or PAH includes the fact that TTM can “teach” pluripotent stem cells to behave and turn them back into normal vascular wall cells, instead of endlessly proliferating. Research has shown that progressive adaptation of human embryonic self-renewing stem cells to their culture conditions does occur, and we expect the conditions created from TTM will turn these stem cells back into normal vascular wall cells.

Various embodiments of the present disclosure include methods for cancer and/or PAH therapy, which utilize TTM in an extended release design as TTM alone, or TTM combined with ATX or a 5-lipoxygenase inhibitor such as DEC or Zileuton, with or without ATX, or inclusion of LEAPS peptide heteroconjugates, tart-butylhydroquinone, or SFN. Embodiments also include a cytokine mixture in a method for pre-sensitizing cancer prior to a therapeutic treatment with TTM and DEC or Zileuton. In embodiments, the cytokine mixture is a serum-free and mitogen-free mixture containing specific ratios of cytokines such as IL-10, TNF-α, IFN-γ, and GM-CSF to Interleukin 2 (IL-2), which is effective in inducing cancerous cells to enter a proliferative cell cycle phase thereby increasing their vulnerability to TTM and DEC or Zileuton therapy. One such cytokine mixture is Leukocyte Interlukin Injection (LI) or MULTIKINE®, which is used with the TTM and DEC combination or Zileuton. In further embodiments, treating patients with TTM and DEC when pre-sensitized with SFN allows for isolating a cell population during its vulnerable cell cycle phase where cells are specifically vulnerable to damage. Unlike chemotherapy, applying TTM and DEC (which are not toxic drugs and have far fewer side effects) makes this therapy ideal for treating cancer and/or PAH. According to various embodiments, the co-drugs to TTM are in an extended release formulation, or TTM is the only drug that is extended release and the co-drugs are not in an extended release formulation.

In addition to TTM alone, in some embodiments the therapy contains at least one other active agent, or co-drug, in combination with TTM.

For example, possible co-drugs include inhibitors of the 5-lipoxygenase enzyme (5-LO), which drives inflammation and controls cell growth, such as DEC and zileuton. The 5-LO protein is expressed more in the tumor tissue and in metastases in patients with cancer which has cell growth that has a similarity to cancer.

Thus, in various embodiments, TTM and a 5-LO inhibitor or a LTA4 inhibitor work synergistically for cancer and PAH. In various embodiments, ATX works synergistically for cancer and PAH as well. While a TTM salt is expected to induce anoikis (inducing death of tumor vessel endothelial cells), inhibition of 5-LO is expected to decrease inflammation and inhibit 5-LO-dependent cell growth. The 5-LO enzyme acts as an activator of gene expression. 5-LO leads to the production of leukotriene C4, which is the first and well-established action of 5-LO. Thus, inhibiting 5-LO would also inhibit leukotriene C4 synthesis. A second action of 5-LO is a non-enzymatic function of binding to the 5-LO activating protein (FLAP) on the envelope of the cell nucleus. Fitzpatrick and Lepley showed in 1998 that 5-LO co-precipitated with a subunit of the transcription factor NF-kappaB when they examined nuclear extracts (Archives of Biochemistry and Biophysics, 1998, 356(1) 71-76). NF-kappaB controls the expression of genes encoding several LTB4 inflammatory mediators. Thus, 5-LO, by binding to NF-kappaB in the cell nucleus, could activate transcription of a number of genes in control of cell growth and genes encoding inflammatory mediators such as IL-1beta and IL-6—and also VEGF. As a result of 5-LO inhibitor treatment, there would be a reduction in vascular inflammation and perhaps stem cell reprogramming leading to halting of tumor growth and metastatic dissemination and assist disease arrest. LTB4 is another important chemotactic leukotriene that is a product of the enzyme leukotriene A4 hydrolase—which is downstream from 5-LO. Because effective inhibition of the 5-LO would also block LTB4 production, it is expected that 5-LO inhibitors in the treatment of cancers would also target LTB4-dependent pathomechanisms. By adding ATX in combination with DEC, the activity of NF-kappaB is decreased to a greater extent than achieved with TTM or DEC, and via the blockade of MAPK, the action of leukotrienes is inhibited.

In some embodiments, cancer in a patient, or a patient with PAH, is treated with a therapeutically effective amount of TTM in an enteric oral formulation dose with or without an extended-release formulation.

In some embodiments, cancer in a patient, or a patient with PAH, is treated with a therapeutically effective amount of a combination of TTM in an extended-release formulation and at least one 5-LO inhibitor. In some embodiments, cancer and/or PAH treatment includes the administration of a therapeutically effective amount of ATX and one or more of the 5-LO inhibitors DEC or zileuton, in combination with a therapeutically effective amount of TTM. In various embodiments, these 5-LO inhibitors and ATX are or are not in an extended release formulation.

In some embodiments, cancer and/or PAH treatment includes the administration of a therapeutically effective amount of ATX and one or more of the 5-LO inhibitors DEC or zileuton, in combination with a therapeutically effective amount of TTM. In various embodiments, these 5-LO inhibitors and ATX are or are in an oral dose, in either and tablet or capsule form that is enterically coated.

In some embodiments, cancer in a patient, or a patient with PAH, is treated with a therapeutically effective amount of a combination of TTM, in an enteric coated tablet, and at least one 5-LO inhibitor. In some embodiments, cancer and/or PAH treatment includes the administration of a therapeutically effective amount of one of the 5-LO inhibitors DEC or zileuton in combination with a therapeutically effective amount of TTM.

In some embodiments, cancer in a patient, or a patient with PAH, is treated with a therapeutically effective amount of a combination of TTM, and with or without at least one 5-LO inhibitor and SFN.

In some embodiments, a patient with cancer and/or PAH is treated with a therapeutically effective amount of a combination of TTM, and with or without at least one 5-LO inhibitor and ATX.

In some embodiments, a patient with cancer and/or PAH is treated with a therapeutically effective amount of a combination of TTM, either in an extended release formulation or without an extended release formulation, and administered together with one or more of the following: a 5-lipoxygenase inhibitor, a leukotriene receptor blocker such as Montelukast, LY29311, SFN, or tert-butylhydroquinone.

In terms of administration, some embodiments concerning the administration of both the TTM and the co-drug is in a single dose form or composition, and in other embodiments the TTM and co-drugs are administered in separate compositions. In some embodiments, the TTM, with or without a co-drug, is administered to the patient at a dose of 20 mg to 300 mg/day. In embodiments, the dose is adjusted to produce a target ceruloplasmin level in the patient of 50% of its normal value. According to embodiments, this target ceruloplasmin level is 15-17 mg/dl of plasma.

According to various embodiments, the compositions contain pharmaceutically acceptable carriers and/or excipients. In various embodiments, the compositions are in an intravenous form or an oral form, such as a tablet, a microtablet, or a capsule. In some embodiments, the TTM is in a composition of an oral form, and the co-drug is in a composition of an intravenous or inhalable form. For compositions containing TTM, with or without the co-drugs, in some embodiments specific carriers and/or excipients are added to provide a delayed release of the TTM in the gastrointestinal tract after passage through the stomach. Specifically, the carriers and/or excipients are selected to facilitate protection of the TTM against destruction by gastric acid and enabling optimal intestinal uptake and absorption. For example, in some embodiments the oral forms of the composition include an enteric coating of the tablet or capsule or include a delayed release formulation and composition.

### Examples: Embodiments of Enteric Capsules

TTM degrades rapidly in stomach acid and dosing patients to date in various drug trials over the years with TTM has always involved administering a proton pump inhibitor (PPI) in advance of swallowing a TTM pill. This is due to the well-known fact that the stomach acid breaks down the TTM quite rapidly before it can reach the intestinal tract and be absorbed by the body.

The concept of administering TTM in this manner is that the stomach acid has been somewhat reduced by the proton pump inhibitor and the TTM can then pass on to the intestine without losing potency, thereby insuring TTM's bio-availability. The first problem with this method is that each person is different; the PPIs have a half-life and the timing of controlling the acidity of the stomach varies for each person. The TTM therefore will degrade differently in each person, accounting for a variable bioactivity of the TTM from patient to patient. Further, compliance to a regime of taking a PPI in advance of the TTM is difficult for some patients, and often such treatment protocols are not followed. Note that TTM degrades substantially in a lower pH acidic solution yet less so in higher pH solution. The second problem with this method is the TTM dissolves in the stomach and gives off sulfur smelling gas. This is known as “sulfur burp” and patients do not enjoy this side effect, discouraging the use of this drug.

PPIs inhibit the gastric H,K-ATPase by covalent binding, so the duration of their effect is longer than expected from their levels in the blood. However, PPIs cannot inhibit all gastric acid pumps with oral dosing because not all pumps are active during the 90-minute half-life of the PPI in the blood. Because PPIs have a short half-life, only 70% of the pump enzymes are inhibited. It takes about 2 to 3 days to reach steady state inhibition of acid secretion. The pump protein has a half-life of about 54 hours in the rat (and probably in humans). Thus, about 20% of pumps are newly synthesized over a 24-hour period, and there may be greater pump synthesis at night than during the day. In addition, bedtime administration of PPIs will not add to inhibition of nocturnal acid breakthrough, because the drug will have disappeared by the time nighttime acid secretion is evident. Assuming that about 70% of pumps are activated by breakfast and that the PPI is given 30 to 60 minutes beforehand, it can be calculated that steady state inhibition on once-a-day dosing is about 66% of maximal acid output. Increasing the dose has virtually no effect once optimal dosage has been reached. Increasing the dose frequency does have some effect; a morning dose and an evening dose before meals results in about 80% inhibition of maximal acid output.

In the manufacture of TTM, the initial TTM particles are larger crystals, so large sometimes, that they are crushed and screened to assure that TTM will flow properly for filling capsules and that the bioavailability is high enough (large particles dissolve slowly). This crushing process creates very small sizes that will degrade quickly in acid, and depending on the ratio of TTM to water, will dissolve quickly in water. These small sizes are vulnerable to destruction and are one explanation of why some patients medicated with TTM do not respond to the TTM therapy or do not respond to the degree required. This is simply because not all of the acid is neutralized and since many of the TTM particles are small and susceptible to immediate destruction, they are not therapeutically active.

To optimize bioavailability is important, and thus, small particles must survive the environment of the stomach and dissolve in the intestine. We propose that differences in treatment outcomes can to some extent be explained by the differences in bioavailability that can be shown by measuring serum ceruloplasmin. TTM is a copper chelator, and if the TTM is bioavailable and copper levels have been lowered, the ceruloplasmin serum levels decrease. In some clinical studies the treatment success correlated with the achieved low ceruloplasmin levels. For one study, lowering serum ceruloplasmin levels (ideally to 50% of normal) was not achieved in a number of patients. One explanation for the failure to lower serum ceruloplasmin levels can be a lack of bioavailability due to TTM destruction by stomach acid. Further, as TTM dissolves in water, and even in an acid reduced stomach (higher pH) there is enough water to dissolve TTM, creating the potential to destroy the TTM by the remaining acidity, especially for the very small particles that dissolve easily.

There have been attempts to address this problem other than adding a proton pump inhibitor, and one study showed that smaller TTM crystals were destroyed immediately by the stomach acid and much larger TTM crystals have a higher level of survival, as some percentage of each crystal is destroyed, but due to the lower specific surface area of a larger size of a TTM crystal, there is a remaining portion of the crystal passed from the stomach to the intestine for the body to absorb this remaining TTM.

Some literature has discussed the use of an enteric coating for administering TTM, however nothing describes how such an enteric coating for TTM is supposed to work and no commercial enterically coated TTM capsules have been developed or offered commercially for delivering TTM in an enteric coating that is not destroyed while in the stomach. Empty enteric capsules to be filled with Active Pharmaceutical Ingredients (API) are common and are commercially available, however, they are not effective in preventing the stomach's acid from attacking and destroying the TTM as enteric capsules are designed to slowly dissolve, delaying the release of the API, however as they dissolve, the TTM is subjected to acid that degrades the TTM. The inventors experimented also with liquid spray on coatings applied to enteric and non-enteric capsules and found the seepage of acid to the TTM was also present. The same occurred to enteric coatings applied to a non-enteric tablet.

Enteric capsules, such as VCAPS® provided by Lonza for any drug manufacture to fill with their API, meet the requirement of an industry standard enteric coating and show a diffusion rate of 0.5% for each 15 minutes, with an accelerated diffusion increase as time passes. For many drug products added to these enteric capsule products, during the first 120 minutes, the capsule body does not seal 100% and the drug slowly escapes from the capsule, as this is the design of such capsules. While for some drugs, such as Acetaminophen, this is an acceptable solution, this is not the case for TTM that is highly stomach acid sensitive.

The present inventors have determined that while the designed disintegration properties of an enteric capsule remain stable for 2 hours and the designed enteric capsule may have a diffusion rate below 7.5% in the first two hours, the inventors research confirmed in fact stomach acid or moisture from the stomach does enter the capsule, and the enteric interior capsule walls become wet, thereby subjecting the TTM to the acid or moisture, causing rapid degradation of the enteric capsule from the chemical reaction with the moistened TTM.

The present inventors have determined that the TTM releases H₂S gas when a small amount of stomach acid or moisture enters the enteric capsule, not only causing degradation of the TTM, but also the moisture seeping through the capsule causes TTM to release H₂S as part of the degradation process, which then comes in contact with the inside of the enteric capsule. This dissolves the enteric capsule from inside the capsule and leads to a catastrophic capsule failure within 15 to 30 minutes.

The present inventors attempted to prevent this failure by applying additional enteric coatings, and even with additional coating, after one hour it was found that the TTM was still leaking out, providing evidence that while an enteric capsule with additional enteric coatings applied will remain intact, stomach acid and moisture will still get through an enteric coating. This is not a preferred solution as the TTM is getting wet inside the capsule and degrading, creating the problem of not knowing how much TTM will be left to be absorbed by the patient after the tablet leaves the stomach. Such a reaction reduces the amount of drug being available to the patient, thereby reducing the drugs intended effect due to less TTM available.

The present inventors undertook a study to determine the size of the TTM particles after a crushing and screening process using a 70-mesh screen that would allow a maximum particle size of 210 micrometers. The size range was as follows: 10% under 13.3 μm; 50% under 44.5 μm; 90% under 122 μm.

Using this data set, the present inventors studied solubility in water and found that 60 mg in 300 ml dissolved fully over time, whereas 160 mg in 300 ml of water did not fully dissolve and a clump remained. This is not unsurprising, as saturation will occur in fluids as a known principle and does illustrate enough fluid is required to be available to dissolve the administered TTM to create optimal bioavailability. Further, larger particles with larger surface area would be expected to remain after the smaller particles dissolve.

This result, when applied to the intestine, knowing that the intestine does not have a larger content of water at any one spot, and contracts to mash, mix, and move contents contained within it through a process called peristalsis, creating wave-like movements that push the contents of the canal forward, means that initially upon entering the intestine, not all the TTM will dissolve and be bio-available.

As TTM is a water-soluble crystal, smaller particles have higher specific surface area and thus have faster bioavailability than larger crystals.

As the intestine eventually will process the TTM dose as it releases from the tablet or capsule with enough water content to dissolve all the TTM in time, having a portion of small particles to create a high bio-availability is important for faster delivery after the tablet or capsule is ingested, and is highly desirable in certain disease states where delivery time to blood is critical. For this reason, ideally 50% of the TTM particles are under 44 μm, for the first immediate dissolution as the enteric capsule dissolves in the intestine.

An aspect to solving this problem of TTM destruction in the stomach acid is to realize that the enteric capsules and coatings will absorb some moisture and while enteric capsules and coatings are designed to prevent the drug release in the stomach, some moisture can seep through an enteric capsule or enteric tablet, through an enteric coating, to then be in contact with the TTM, causing a chemical reaction that destroys the enteric protection from within the tablet or capsule.

Another aspect to enhance bioavailability of the TTM is to assure that small particles of TTM survive to be released in the intestine, ideally over 90% of the molecules are smaller than 122 μm, and no less than 50% smaller than 122 μm, when the enteric capsule releases into the intestine.

According to various embodiments of the disclosure, to solve the stomach acid destruction of the TTM in an enteric capsule, the TTM is isolated from the inside surfaces of the enteric capsule or tablet design and moisture is prevented from contacting the TTM and thereby destroying the integrity of the enteric capsule from the inside. This protection only has to last for a long enough time for the TTM to pass from the stomach to the intestine, generally about 2 hours.

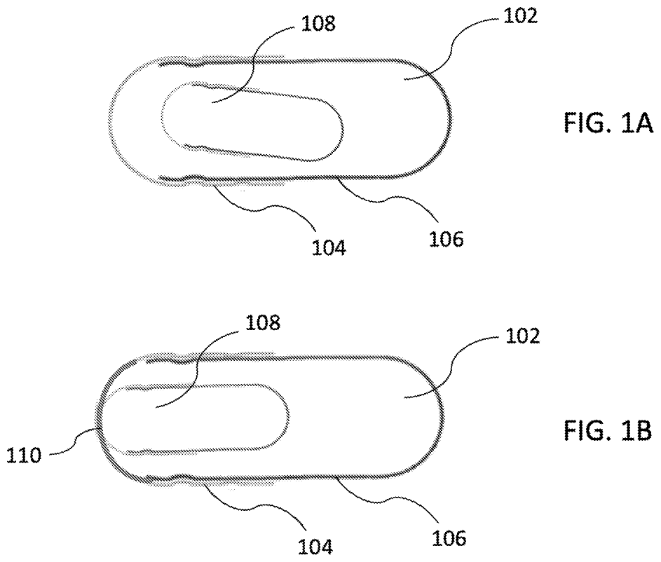

According to various embodiments, the TTM is encapsulated inside a first capsule, and the first capsule in encapsulated inside a second capsule. In one embodiment, illustrated in FIG. 1A and FIG. 1B, the enteric capsule 102 includes a body 104 and a cap 106, in size 0 or size 00. These are larger size capsules, and a smaller size 1, 2, 3 or 4 capsule 108 is filled with TTM or with TTM and a filler and inserted inside the enteric coated larger size 0 or size 00 capsule. In embodiments, the filler is in powder form or is an oil that does not mix with water. Different lipids and fats, synthetic and natural polymers, natural lipophilic substances, other water-absorbing substances, inorganic fillers, such as silica or colloidal silica, or other materials can be used as long as the fillers do not swell significantly upon contact with water and are stable under conventional storage conditions. In some embodiments, the filler is an oil and further includes one or more co-drug, such as ATX and/or DEC. Other sizes then 0 or 00 can be the outside second capsule provided the inside first capsule containing the TTM can fit in the second capsule loosely.

According to embodiments, one criterion of the smaller capsule inside the larger capsule is that the smaller capsule is of low water content so as not to react with the TTM and slowly dissolve with a minor amount of moisture, where enough of the capsule remains intact for at least two or more hours when subjected to stomach acid, to prevent the moisture that is entering the enteric capsule from destroying the capsule. This result of “enough of the capsule” remaining intact, is a criterion as this is a measurement of the maximum amount of liquid entering through the outer enteric capsule.

According to embodiments, another criterion of filling TTM in the smaller capsule is that any excipients needed to enhance flow and/or fully fill the capsule when the dosage of TTM is not enough to fill the capsule, the excipient must be of very low or no water content to avoid a reaction with TTM. The inventors determined that while mannitol could be used as a filler, in some embodiments, blending mannitol with colloidal silicon dioxide enhances the ability to absorb moisture, and that colloidal silicon dioxide allowed very good flow into the smaller capsule.

According to various embodiments for filling a capsule, TTM is blended with only colloidal silicon dioxide, as colloidal silicon dioxide provides a needed benefit of flow enhancement into the capsule and moisture uptake that will increase the stability of the TTM.

Moisture adsorbs on the surface of colloidal silicon dioxide by the formation of siloxane bonds (≡Si—O—Si—) and silanol groups (≡Si—OH), which are capable of forming numerous hydrogen bonds, thus affecting the collective forces between individual particles, improving the prevention of moisture that can otherwise react to the TTM. The prevention of such moisture or acid ensures the stability of the TTM.

Gelatin capsules are by design made with 13% to 16% water content and dissolve quickly in moisture and are thus less suitable for encapsulating the TTM, whereas hydroxypropyl methylcellulose (HMPC) capsules are available with a low moisture content of 2 to 3.5%. HPMC capsules have an inherently low moisture content which allows for the encapsulation of ingredients that are sensitive to moisture and are hygroscopic in nature or react to moisture, such as TTM.

There are two classes of HPMC capsules—the standard HPMC with a water content of 3% to 8%, and low moisture-content HPMC with a water content of 2% to 3.5%. In various embodiments, the low moisture content HPMC capsules will not easily impart moisture to the TTM that would cause H₂S to be released and these HPMC low water content capsules absorb the initial moisture that passes though the enteric capsule and then eventually penetrates the HPMC capsule.

Note that while any acid that reaches the TTM will start a degradation process and release of H₂S, the degradation of TTM is fast when exposed to acid. For this reason, the TTM must remain dry while in the stomach. The present inventors analyzed several manufacturers of enteric capsules and determined an ideal capsule was one that provided a minimal amount of moisture seepage, because a low level of moisture seepage, when achieved, did not dissolve the HPMC capsule in a rapid fashion, and prevented most of acid from reaching the TTM.

This innovation is an enteric capsule that limits the moisture contacting the internal capsule to an amount that does not dissolve an HPMC capsule. Not all enteric capsules allow the same amount of acid to seep inside and the enteric capsule selected is designed to be intact for two hours when subjected to stomach acid.

The present inventors also determined that the smaller HPMC capsule would still start to dissolve and wet the TTM earlier than desired when a surface of the inside smaller HPMC capsule was in direct contact with the inside wall of the larger enteric capsule. To isolate the inside HPMC capsule from the inner wall of the enteric larger capsule, a filler in powder form was added prior to closing the enteric capsule with the HPMC TTM filled capsule inside.

As shown in FIGS. 1A and 1B, the capsule within a capsule design requires a distance between the inner capsule 108 and outer capsule 102 that must be enough to allow the filler to isolate the inner capsule 108 from touching the outer capsule 102. FIG. 1A illustrates an acceptable capsule design in which the inner capsule 108 is completely isolated from the outer capsule 102. FIG. 2B illustrates an unacceptable capsule design in which the inner capsule 108 is not completely isolated. There is a contact point 110 between the inner capsule 108 and outer capsule 102.

Another selection criterion for the size of the outer enteric capsule and the inner capsule is that the size differential between the two capsules is enough such that an adequate filler barrier can be placed between the two capsules to assure that the wetness from the outer capsule, when such capsule absorbs water or stomach acid, making the inside of this enteric capsule wet, significantly reduces contact with the inner capsule. In some embodiments, one size that is effective is a size 00 outer enteric capsule with a size 4 inner capsule. Other embodiments utilize a size 000 outer enteric capsule. Some embodiments utilize a size 0-size 5 inner capsule.

Another innovation of the present disclosure is the vibration of the capsule after assembly that allows the inner capsule to float within the filler and no part of the inner capsule contacts directly the inner wall of the larger enteric capsule. The present inventors found that without such vibration, for some of the filled capsules, the inner capsule still contacted directly the inner surface of the outer enteric capsule, whereby the inner capsule started softening and transferred stomach acid to the TTM, with about 30%-50% of the TTM becoming wet and starting to degrade when submersed in simulated stomach acid for two hours.

Another innovation of the present disclosure is the selection of a powder or filler or adsorbent, based on silica, that had two key properties. One property is to absorb moisture or acid as this moisture or acid seeps through the outer layer. A second property is to allow the inner capsule to move away from the wall of the outer capsule and “float” while being vibrated to a position where all sides of the inner capsule contact the filler and not the inner wall of the larger enteric capsule. Note that it is important to select non-swelling materials for the filler as otherwise the outer capsules might be destroyed.

Another innovation of the present disclosure is the selection of a powder or filler, and that while colloidal silicon dioxide may be an ideal moisture absorbent, in some embodiments it is too light to flow well and does not easily fill the capsules. In some embodiments, mesoporous dicalcium phosphate is blended with colloidal silicon dioxide, and an ideal result of preventing the inner capsule to stay dry for at least two hours or more was achieved. In formulations that use solely mesoporous dicalcium phosphate, the absorption properties of mesoporous dicalcium phosphate are not enough to prevent the inner capsule containing the TTM from becoming in contact with the acid that was seeping through the enteric capsule.

The foregoing disclosure is a design to assure that the TTM has at least two hours before becoming wet, as the human stomach generally passes food to the intestine between 30 minutes to two hours.

The present inventors were also able to substitute the larger outer capsule with an outer HMPC capsule, such HMPC capsule being either a standard capsule (i.e., 3-8% water content) or a low water capsule (2-3.5% water content), and applying enteric coatings and seals to this larger non-enteric capsule, thereby creating an enteric capsule, and found this could be made to work.

One alternative embodiment to using the internal HMPC capsule—isolated from the inside of the larger outer capsule with powder added to isolate the internal capsule or tablet—is to replace the internal HMPC capsule with an enteric capsule. This will slow the transfer of moisture and provide more time for the TTM to leave the stomach dry.

Another alternative embodiment is to replace the inner HMPC capsule with an oil filled capsule. In this embodiment, the TTM is inside of this inner capsule in an oil. A benefit of this approach is that the TTM is then protected from oxidation when suspended in oil and should any water seep in through the wall of the inner capsule, the oil protects the TTM from the water or acid. In some embodiments, ATX is optimally provided in oil, such as olive oil. ATX in oil, between the inner capsule and the outside enteric capsule serves to isolate the internal capsule containing TTM.

In another embodiment of the oil filled capsule that replaces the HMPC capsule, an enteric coating is applied to the outside of the inner capsule to protect the TTM from any water that seeps into this capsule.

In another embodiment, the powder inserted between the inner capsule and outer capsule is a mixture of different components, e.g., a co-drug and fillers and/or absorbents. In some embodiments, the co-drug is DEC. In embodiments, the DEC is up to a dose of 300 mg, or less, and can be mixed with adsorbent, such as silica, magnesium oxide/carbonate, kaolin/bentonite or other adsorbing materials or with non-swelling fillers, such as di-calcium phosphate, lactose, sugar-alcohols (such as sorbitol, xylitol and mannitol) and others. In embodiments, the DEC is substituted with ATX. In embodiments, the DEC and ATX are combined together as a substitute for DEC. In various embodiments, SFN is also included in the formulation with DEC or DEC and ATX or alone.

In some embodiments, the internal capsule is substituted with enterically coated mini-tablets mixed with adsorbent and placed in the outer capsule. In some embodiments, a compressed TTM tablet is enterically coated and placed inside the larger enteric coated capsule.

In an embodiment of an enterically coated TTM tablet or mini-tablet, the TTM is compressed with a non-reactive binder or compression aid, and is then coated with an enteric coating layer, and potentially, with a barrier-agent layer. Coating occurs via drum or fluid bed spray coating, dip coating, or spray congealing, or other coating approaches. The barrier-agent layer improves resistance to diffusion of moisture through the enteric film, i.e., from reaching the TTM as the enteric coating starts to dissolve. The barrier-agent layer is such that it either absorbs water, or slowly dissolves (e.g., a functional controlled-release polymer), or may be a lipophilic (hydrophobic) coating substance, e.g., lipids and other hydrophobic excipients, preventing water or stomach acid from reaching the TTM for 2 hours. In some embodiments, the barrier layer also contains a co-drug, such as DEC, or ATC, or both ATC and DEC, or another co-drug.

In another embodiment, the TTM is packaged alone in an enteric tablet or capsule of a design described herein to protect the TTM from acid seeping through the enteric tablet or capsule, and the DEC and/or ATX is packaged in a separate tablet or capsule. In some embodiments, both the DEC and the ATX are enclosed in an enteric tablet or capsule. It is known that DEC is a citrate salt, and as such, large doses can cause nausea. By administering in an enteric tablet or capsule, this nausea is avoided. ATX is highly lipophilic, and presently ATX is being sold as a gel capsule that contains ATX combined with olive oil or other vegetable oils. This combination results in a greater stability of the ATX compound and also in better bioavailability. It has been recommended to take this capsule together with a fat-containing meal to increase bioavailability, largely because ATX, being lipophilic, combines or dissolves in lipids or fats, which protects ATX from the stomach acids. By providing ATX in an enteric capsule, the bioavailability increases. In an embodiment of a DEC and ATX pill, tablet, or capsule, both drugs are combined in an enteric capsule.

In one embodiment, the DEC is in an internal tablet or capsule, ideally in an enteric capsule or tablet form, surrounded by ATX in oil, and the ATX is filled and captured by an outer capsule or gel cap that, such outer capsule or gel cap that may or may not be enterically coated. In this embodiment, the DEC will not release until after the ATX releases.

In another embodiment the DEC and ATX are combined in one capsule that may or may not be enterically coated.

In another embodiment, an outer and larger enteric capsule contains a smaller capsule that contains ATX, the smaller internal capsule with ATX is filled with oil to increase the bio-availability, and this smaller internal capsule with ATX is inserted into a larger capsule. The larger capsule is filled with DEC in a powder form, with or without suitable excipients that may be needed to assure a full fill and adequate flow, between the outer enteric capsule and the ATX internal capsule. The benefit of this design of an ATX/DEC capsule is that the ATX is protected from the stomach acids and the DEC is protected as well, released after the stomach, to avoid the nausea that is a known side effect of DEC in larger doses.

In some embodiments, a combination tablet of DEC and ATX is administered in a composition containing pharmaceutically acceptable carriers and/or excipients. In embodiments, the compositions are administered in an oral form, such as a tablet, a microtablet, a capsule, or a sachet. In some embodiments, the combination is a composition of an oral form with specific carriers, matrix compounds, and/or excipients that provide a delayed release of the two drugs in the gastrointestinal tract after passage through the stomach. In general, the carriers, matrix compounds, and/or other excipients are selected to facilitate extended and controlled release of the DEC and ATX, enabling optimal intestinal uptake and absorption, guaranteeing stability for storage, and possibly minimizing risk of alcohol-related dose dumping. Moreover, the carriers, matrix compounds, and/or excipients are selected such that destruction by gastric acid is avoided. For this objective, suitable coating materials for enteric coating are applied. In some embodiments, the oral forms of the composition include an enteric coating of the tablet, micro-tablet, capsule, or of individual pellets and beads in the capsule. Also, in some embodiments, multiple coating layers are applied, e.g., a coating layer for enteric coating and an extended release coating layer. Also, in some embodiments, a layer is added to avoid burst release. Also, in some embodiments, different dosage forms are combined, such as capsules in capsules, or mini-tablets in capsules. In embodiments, the capsules and mini-tablets are combined with a filler and an adsorbent to minimize exposure to liquid water and stomach acid.

In various embodiments, the oral coating is an extended-release coating, whereby the extended-release coating creates a stable administration of the DEC/ATX tablet combination whereby it is released over a period of time creating a long- or extended-release of TTM and eliminating the need for taking several tablets over a given time period.

In embodiments, the extended release is designed as matrix tablets or pellets in a capsule where either a hydrophilic or lipophilic polymer is used as carrier or a lipid matrix.

In various hydrophilic matrix system embodiments, DEC and ATX are dispersed throughout a polymer matrix of hydrophilic material. The rate of drug release is controlled by both diffusion and erosion. When water is absorbed by the matrix, the matrix swells and the polymer on the surface of the tablet hydrates. DEC and ATX dissolves and is released by a combination of diffusion out of the matrix, through the gel layer, and as a result of the erosion of the matrix itself. In various embodiments, the matrix system is enterically coated to inhibit destruction of the copper chelator by stomach acid. Alternatively, in some embodiments, the matrix based system is put in an enteric capsule, where the capsule is either made from an enteric material, or is coated and sealed enterically.

In various hydrophobic matrix system embodiments, DEC and ATX are dispersed throughout a polymer matrix of inert hydrophobic material, either polymer or lipid. In embodiments, the hydrophobic matrix undergoes no or minimal swelling on contact with water. When water enters the matrix, DEC and ATX dissolves and is predominately released by diffusion out of the matrix. In various embodiments, the matrix system is enterically coated to inhibit destruction of the copper chelator by stomach acid. Alternatively, in some embodiments, the matrix based system is put in an enteric capsule, where the capsule is either made from an enteric material, or is coated and sealed enterically.

In another embodiment, suitable polymers or lipid carriers are used for a hot-melt extrusion process to make the extended-release material, which can further be processed into tablets, micro-tablets, pellets, or beads. One or more coating layer(s) adds enteric protection and avoids burst release. In embodiments, another barrier is coated onto the delivery form (either on top or below the enteric coating). This barrier layer is designed such as to improve resistance to diffusion of moisture through the enteric film, i.e., from reaching the copper chelator as the enteric coating starts to dissolve. The barrier agent layer is such that it either absorbs water, or slowly dissolves (e.g., a functional controlled-release polymer), or is a lipophilic (hydrophobic) coating substance, e.g., lipids and other hydrophobic excipients.

In various embodiments, the DEC/ATX combination is packaged in an extended release system by designing a reservoir system, whereby a core containing DEC or ATX is surrounded by an insoluble polymer membrane of suitable extended release polymers. In embodiments, the DEC/ATX combo is contained in a single core. In embodiments, the DEC/ATX combo is subunits, such as beads, pellets, or mini-tablets, containing the drug. In embodiments, a coating layer adds enteric protection and avoids burst release.

In embodiments, DEC and ATX are packaged in a capsule with multiple different subunits, such as micro-tablets, pellets, or beads, with different release characteristics allowing for a multimodal IR (immediate release) plus extended release (ER). The capsule is enterically coated or the subunits are enterically coated. In embodiments, an additional moisture-diffusion-limiting barrier layer minimizes acid-related destruction of the ATX and nausea caused by DEC otherwise releasing in the stomach.

According to various embodiments, the DEC or ATX is packaged in an extended-release system using an osmotic-release system of a drug-containing core surrounded by an insoluble but semipermeable membrane capsule or coating. This membrane contains an orifice through which the soluble drug is forced by osmotic pressure that builds up inside the capsule on contact with water. This delivery system is enterically coated to ensure that no DEC or ATX is released in the stomach.

FIG. 2-FIG. 12 illustrate oral formulations of TTM according to various embodiments of the present disclosure. In FIG. 2, a tablet 202 has a core that contains TTM 204 and has an enteric coating 206, along with an antacid coating 208. The TTM 204 is combined with excipients (e.g., disintegrants and binders) that allows the TTM to release easily after it dissolves in the intestine.

In FIG. 3, a tablet 302 has a core that contains TTM 304, an enteric coating 306, and an antacid coating 308. The tablet 302 further contains ATX and/or DEC 310 with or without needed binders and disintegrants, providing a barrier to protect the TTM 304 from stomach acids seeping through the antacid coating 308.

In FIG. 4, a tablet 402 has a core that contains TTM 404 and has an enteric coating 406. The tablet 402 further contains ATX and/or DEC 410. The ATX and/or DEC 410 is combined with an antacid and suitable excipients and disintegrants, providing a barrier to protect the TTM 404 from seeping stomach acids and to help neutralize the acid.

In FIG. 5, a tablet 502 has a core that contains TTM 504 and another co-drug 512, such as DEC or another co-drug, along with an enteric coating 506. The tablet 502 also contains ATX 510, combined with an antacid and suitable excipients and disintegrants.

In FIG. 6, a tablet 602 has a core that contains TTM 604 and another co-drug 612, such as DEC or another co-drug, along with an enteric coating 606 and an antacid coating 608. The tablet 602 also contains ATX 610 combined with suitable binder and disintegrants.

In FIG. 7, a tablet 702 has a core that contains TTM combined with another co-drug 714, such as DEC or another co-drug, along with an enteric coating 706 and an antacid coating 708.

In FIG. 8, a tablet 802 has a core that contains TTM 804. The tablet 802 also contains one or more other co-drug 812, such as DEC, ATX, and/or another co-drug, and one or more other co-drug 810, such as DEC, ATX, and/or another co-drug, combined with an antacid and suitable excipients and disintegrants, along with an enteric coating 806.

In FIG. 9, a tablet 902 has a core that contains TTM 904. The tablet 902 also contains one or more other co-drug 912, such as DEC, ATX, and/or another co-drug, and one or more other co-drug 910, such as DEC, ATX, and/or another co-drug, along with an enteric coating 906.

In FIG. 10, an oral formulation includes a multi-layer tablet 1002 with TTM 1004, DEC or other co-drug 1012, and ATX or other co-drug 1010. The multi-layer tablet 1002 also has an enteric coating 1006 and an antacid coating 1008 encapsulating the TTM 1004, DEC or other co-drug 1012, and ATX or other co-drug 1010. In embodiments of the tablet 1002, different layers of the drugs and co-drugs in a suitable form (e.g., particles, nano-particles, micronized particles, pellets, enterically coated pellets) and suitable excipients (e.g., binders, disintegrants, stabilizers) are made in a multi-layer compression process. The multi-layer core is coated first with the antacid coating 1008 and then with the enteric coating 1006.

In FIG. 11, an oral formulation includes a multi-layer tablet 1102 with TTM 1104, DEC or other co-drug 1112, and ATX or other co-drug 1110. The multi-layer tablet also has an antacid coating 1108 encapsulating the TTM 1104, and an enteric coating 1106 encapsulating the TTM 1104, DEC 1112, and ATX or other co-drug 1110. In embodiments of the tablet 1102, different layers of the drugs and co-drugs in a suitable form (e.g., particles, nano-particles, micronized particles, pellets, enterically coated pellets) and suitable excipients (e.g., binders, disintegrants, stabilizers) are made in a multi-layer compression process. The TTM 1104 is first coated with the antacid coating 1108 and then the multi-layer core is coated with the enteric coating 1106.

In FIG. 12, an oral formulation includes a multi-unit pellet system (MUPS) providing a tablet 1202 that includes compressed pellets 1220 of one more drugs and/or co-drugs, such as TTM 1204, ATX 1210, and/or DEC 1212, mixed with suitable tableting excipients (e.g., binders, disintegrants) 1226. In various embodiments, the pellets 1220 have an enteric coating, e.g., 1222 and 1224. In some embodiments, the excipient matrix also includes non-pelletized drugs or co-drugs. The tablet 1202 also has an enteric coating 1206.

In various embodiments of the TTM formulations in FIG. 2-FIG. 12, the antacid comprises one or more of aluminum hydroxide, calcium carbonate, magnesium carbonate, magnesium hydroxide, magnesium trisilicate, sodium bicarbonate, alginate, or a combination thereof, along with appropriate binders and disintegrants. In some embodiments of the TTM formulations in FIG. 2-FIG. 12, the TTM is combined with a filler or binder and disintegrants that allows the TTM to release easily after it dissolves in the digestive system. In some embodiments, the enteric coating further includes a co-drug, such as DEC and/or ATX. In various embodiments, the TTM formulation is in the form of a tablet or a capsule and can have any shape.

According to various embodiments of the manufacturing process, direct compaction is used to manufacture the tablets. In some embodiments, a combination of DEC and suitable excipients and ATX and suitable excipients are fed directly to a tablet press, producing a tablet with each drug using standard extended-release excipients (such as a HPMC, PEO or Eudragit RL, RS, cross-linked PVA) and/or more advanced excipients including polymer mixtures. Moreover, other excipients for lubrication, stabilization, coloring, taste-masking, or for use as fillers and binders may be added in the powder mixture. Good flowability of the powder and low tendency for segregation of the powder mixture are desired. The process can be carried out in batch or in continuous mode.

In another embodiment, roller compaction followed by milling and screening is applied to make granules which are then mixed with lubricants and other excipients for tableting. In an embodiment, DEC and ATX is combined and standard extended-release excipients are used (such as a HPMC, PEO or Eudragit RL, RS, cross-linked PVA) and/or more advanced excipients including polymer mixtures. Moreover, other excipients for stabilization, coloring, taste-masking or for use as fillers and binders may be added in the powder mixture. The process can be carried out in batch or in continuous mode.

In another embodiment, wet granulation, e.g., via massing and screening or high-shear wet granulation or twin-screw wet granulation, with solvents that do not react to or otherwise degrade the ATX or DEC can be used to make granules, followed by a drying process to produce dry granules which are used for tableting. The solvents used cannot contain water. Standard extended-release excipients may be used (such as a HPMC, PEO or Eudragit RL, RS, cross-linked PVA) and/or more advanced excipients including polymer mixtures. Moreover, other excipients for stabilization, coloring, taste-masking or for use as fillers and binders may be added in the powder mixture. The process can be carried out in batch or in continuous mode.

In another embodiment, hot-melt extrusion is applied where powders or powder mixtures containing ATX and DEC, a matrix material for extended release, and other excipients are fed to a hot-melt extruder. The extruded strand is cooled and milled, or is directly processed into pellets and beads or to tablets via calandering. In embodiments, milled material is mixed with suitable excipients and is tableted on a tableting machine. The process can be carried out in batch or in continuous mode.

In embodiments, for coating the DEC/ATX tablets, standard or advanced drum coaters are used that also can be used to carry out multiple coating steps. In embodiments, for coating of micro-tablets, pellets, and beads, fluidized bed coaters are used. Sprayed solutions or suspensions contain the required polymers for enteric coating, or extended release and suitable colors, pigments, surfactants, plasticizers and other components. Alternatively, spray congealing or dip coating is applied.

In some embodiments, cancer and/or PAH in a patient is treated by administering TTM with a therapeutically effective amount of DEC and ATX. The amount of DEC and ATX administered to the patient is individualized. According to various embodiments, the therapeutically effective amount of the ATX is in a range of about 5 mg to 30 mg of ATX/day.

The amount of ATX is adjusted according to the level of decrease in oxidative stress markers (e.g., malondialdehyde and isoprostane) and the decrease in markers of inflammation (e.g., CRP and IL-1). According to various embodiments, the amount of DEC administered to the patient is in a range of about 100 mg to 250 mg per dose up to about 1,000 mg per day.

According to various embodiments, the DEC and ATX are administered in a composition containing pharmaceutically acceptable carriers and/or excipients. In embodiments, the compositions is administered in an oral form, such as a tablet, a microtablet, a capsule filled with pellets or powder, or a sachet. In some embodiments, the DEC and ATX are in a composition of an oral form with specific carriers, matrix compounds, and/or excipients that provide a delayed release of DEC and ATX in the gastrointestinal tract after passage through the stomach. Such matrix materials include, but are not limited to, hydroxypropyl methylcellulose (HMPC), gums, alginates, lipids of various compositions, polyvinyl acetates, polyvinylpyrrolidones, methacrylate copolymers, polyethylene glycols (PEG)/polyethylene oxides (PEO), combinations thereof, and others. In general, the carriers, matrix, and/or other excipients are selected to facilitate extended (sustained) and controlled release of the copper chelator, enabling optimal intestinal uptake and absorption, to guarantee stability for storage and possibly minimizing risk of alcohol-related dose dumping. Moreover, the carriers, matrix and/or excipients are selected such that destruction by gastric acid is avoided. For this objective, suitable coating materials for enteric coating are applied. In embodiments, such a coating is applied externally on the tablet or on drug-containing pellets individually. For example, in some embodiments, the oral forms of the composition include an enteric coating of the tablet, micro-tablet, capsule, or of individual pellets and beads in the capsule. Also, in embodiments, multiple coating layers are applied, e.g., a coating layer for enteric coating and an extended release coating layer. Also, in embodiments, a layer is added to avoid burst release. In embodiments, enteric coatings contain typically pH-sensitive polymers or particles, such as, but not limited to, cellulose acetate phthalate, cellulose acetate trimellitate, shellacs, polyvinyl acetate phthalate, hydroxy-propylmethylcellulose phthalate, hydroxypropylmethylcellulose acetate succinate, poly-methacrylic acids, poly-ethyl acrylates, or poly-methacrylates at various mixtures, amylose starches and other starches, dextrins, plant proteins (e.g., zein and others), fatty acids, lipids including modified lipids, waxes, combinations thereof, and other.

According to various embodiments, the oral coating is an extended-release coating, whereby the extended-release coating creates a stable administration of the DEC and ATX, whereby it is released over a period of time creating a long- or extended-release of DEC and ATX and eliminating the need for taking several dosages over a given time period. Such extended-release coatings are based on different natural or man-made polymers, such as, but not limited to, ethyl celluloses, hydroxypropylmethylcelluloses, methylcelluloses, hydroxypropylcelluloses, hydroxyethylcelluloses, and sodium carboxy-methylcellulose.

In various embodiments, enteric coatings and extended release coatings layers are external and/or internal to the tablets, beads or pellets and can be combined to protect from acidic stomach content and to create a controlled release profile over an extended amount of time.

In various embodiments, the extended release system is designed as matrix tablets or pellets in a capsule where either a hydrophilic or lipophilic polymer is used as carrier or a lipid matrix.

In embodiments of the hydrophilic matrix system, DEC and ATX is dispersed throughout a polymer matrix of hydrophilic materials which have been described above. The rate of drug release is controlled by both diffusion and erosion. When water is absorbed by the matrix, the matrix swells and the polymer on the surface of the tablet hydrates. DEC and ATX dissolves and is released by a combination of diffusion out of the matrix, through the gel layer, and as a result of the erosion of the matrix itself. In formulations, the matrix system is enterically coated to inhibit destruction of the copper chelator by stomach acid. Alternatively, the matrix based system is put in an enteric capsule, where the capsule is either made from an enteric material, or is coated and sealed enterically.

In embodiments of the hydrophobic matrix system, DEC and ATX is dispersed throughout a polymer matrix of inert hydrophobic materials, either polymer or lipid, as discussed above. In this embodiment, the hydrophobic matrix undergoes no or minimal swelling on contact with water. When water enters the matrix, DEC and ATX dissolves and is predominately released by diffusion out of the matrix. In formulations, the matrix system is enterically coated to inhibit destruction of ATX by stomach acid. Alternatively, the matrix based system is put in an enteric capsule, where the capsule is either made from an enteric material, or is coated and sealed enterically.

In another embodiment, suitable polymers or lipid carriers or a combination of both are used for a hot-melt extrusion process to make the extended-release material which can further be processed into tablets, micro-tablets, pellets, and beads. In some embodiments, coating layer(s) for enteric coating and controlled release coating add enteric protection and help avoid burst release. In embodiments, another barrier is coated onto the delivery form (either on top or below the enteric coating). This barrier layer is designed such as to improve resistance to diffusion of moisture through the enteric film, i.e., from reaching ATX as well as the copper chelator as the enteric coating starts to dissolve. The barrier agent layer is such that it either absorbs water, or slowly dissolves (e.g., a functional controlled-release polymer), or is a lipophilic (hydrophobic) coating substance, e.g., lipids and other hydrophobic excipients.

In another embodiment, the DEC and ATX are packaged in an extended release system by designing a reservoir system, whereby a core containing DEC and ATX is surrounded by an insoluble polymer membrane of suitable extended release polymers. In such an embodiment, the DEC and ATX are contained in a single core. In another embodiment, the DEC and ATX are subunits, such as beads, pellets, or mini-tablets, containing the drug. In embodiments, an enteric coating layer adds enteric protection and avoids burst release.

In embodiments, DEC and ATX is packaged in a capsule with multiple different subunits, such as micro-tablets, pellets, or beads, with different release characteristics allowing for a multimodal IR (immediate release) plus extended release (ER). In embodiments, the capsule is enterically coated or the subunits are enterically coated. In embodiments, an additional moisture-diffusion-limiting barrier layer minimizes acid-related destruction of the copper chelator.

In embodiments, the DEC and ATX are packaged in an extended-release system using an osmotic-release system including a drug-containing core surrounded by an insoluble but semipermeable membrane capsule or coating. This membrane contains an orifice through which the soluble drug is forced by osmotic pressure that builds up inside the capsule on contact with water. In embodiments, the semipermeable membrane is made of polymeric materials, such as, but not limited to, cellulose acetate polymers, cellulose esters, cellulose ethers, agar acetates, amylose triacetates, betaglucan acetates, poly(vinylmethyl)ether copolymers, poly(orthoesters), polyacetals and selectively permeable poly(glycolic acid), poly(lactic acid) derivatives, as well as Eudragits. Embodiments of this delivery system are enterically coated to ensure that no copper chelator or ATX or DEC is released in the stomach.

In an embodiment of the manufacturing process, direct compaction is used to manufacture the tablets. In this process, a mixture of DEC and ATX and suitable excipients are fed directly or individually to a tablet press, using standard extended-release excipients (such as a HPMC, PEO or Eudragit RL, RS, cross-linked PVA) and/or more advanced excipients including polymer mixtures. In some embodiments, other excipients for lubrication, stabilization, coloring, taste-masking, or for use as fillers and binders are added in the powder mixture. Such excipients include, but are not limited to, metal soaps such as magnesium stearate, sodium stearyl fumarate, croscarmellose sodium, modified starches, modified lactoses, dextrins, glucose, sucrose, sorbitol dicalcium phosphates, vitamins, colorants, sugar alcohols, crospovidone, polymers and copolymers, silica compounds, silicone or alginates, microcrystalline cellulose, and hydroxypropylcellulose. Good flowability of the powder and low tendency for segregation of the powder mixture are advantageous. Embodiments of the process are carried out in batch or in continuous mode.

In another embodiment, roller compaction followed by milling and screening is applied to make granules, which are then mixed with lubricants and other excipients for tableting. In embodiments, standard extended-release excipients are used (such as a HPMC, PEO or Eudragit RL, RS, cross-linked PVA) and/or more advanced excipients including polymer mixtures. In some embodiments, other excipients for stabilization, coloring, taste-masking or for use as fillers and binders are added in the powder mixture. Embodiments of the process are carried out in batch or in continuous mode.

In another embodiment, wet granulation, e.g., via massing and screening or high-shear wet granulation or twin-screw wet granulation, with solvents that do not to destroy DEC and ATX are used to make granules, followed by a drying process (e.g., via tray drying, fluid bed drying, conveyer belt drying and other methods) to produce dry granules which are used for tableting. In embodiments, standard extended-release excipients are used (such as a HPMC, PEO or Eudragit RL, RS, cross-linked PVA) and/or more advanced excipients including polymer mixtures. In some embodiments, other excipients for stabilization, coloring, taste-masking or for use as fillers and binders are added in the powder mixture. Embodiments of the process are carried out in batch or in continuous mode. In some embodiments, granules are filled in capsules and sachets, or are used for tableting if mixed with an external phase, such as lubricants or binders.

In another embodiment, granules are produced via wet granulation in extruders where a suitable solvent is added and matrix materials and DEC and ATX are added either separately or as a premix. Extruders include twin-screw extruders, radial screw extruders, roll extruders or Koller press extruders. Subsequently to extrusion, granules are dried via methods as described herein. Materials are selected based on required formulation and biopharmaceutical requirements as described herein. In some embodiments, granules are filled in capsules and sachets, or are used for tableting if mixed with an external phase, such as lubricants or binders.

In another embodiment, hot-melt extrusion is applied where powders or powder mixtures containing DEC and ATX, a matrix material for extended release and other excipients are fed to a hot-melt extruder. The extruded strand is cooled and milled, or is directly processed into pellets and beads or to tablets via calandering. In embodiments, milled material is mixed with suitable excipients and is tableted on a tableting machine. Embodiments of the process are carried out in batch or in continuous mode.

In another embodiment, additive manufacturing technology, also known as 3D printing, is applied to make tablets of a desired release profile. For such a process, filaments are manufactured that contain the DEC and ATX and delayed-release matrix materials as described herein. In embodiments, these filaments are made by extrusions process as described herein using different types of extruders including single-screw, double-screw or ram extruders. Filaments are then used to print tablets via thermal technique, such as fused deposition modeling. Alternatively, melt from extruders is directly used to cast tablets via additive manufacturing technology. In embodiments, other additive manufacturing technologies are applied such as powder bed printing, inject printing, VAT polymerization, direct-wise printing, and others. Materials for printing include delayed-release artificial and natural polymers, starches, lactoses, hydroalcohols, lipids, and other natural products.

According to various embodiments for coating the tablets, standard or advanced drum coaters are used, and in some embodiments, also are used to carry out multiple coating steps. In embodiments for coating of micro-tablets, pellets and beads fluidized bed coaters are used. Sprayed solutions or suspensions contain the required polymers for enteric coating, or extended release coatings, and suitable colors, pigments, surfactants, plasticizers, and other components.

According to various embodiments, an extended release tablet contains DEC and/or ATX as co-drugs. Such embodiments contain suitable formulations that minimize chemical interaction between DEC and TTM and ATX. According to various embodiments, formulation techniques are used that separate the chemicals spatially. In some embodiments, this is achieved by making multi-layer tablets with layer 1 containing DEC and layer 2 containing ATX. In some embodiments, pellets of two types are made with methods described herein, with pellet (or bead) type 1 containing DEC and pellet (or bead) type 2 containing ATX. In various embodiments, the pellet (or bead) types are processed in different ways, e.g., by compressing them into one tablet, or coating them separately and embedding them in a matrix material, called multi-unit pellet system (MUPS). In some embodiments, the produced tablets are coated enterically or with extended release coating. In some embodiments, the matrix material of the MUPS is made of extended release matrix materials as described herein. Another embodiment is to prepare multilayer coating of inert bead, having different APIs in different coating layers. Thereafter the beads are filled in capsules with release modifiers. In another embodiment, the DEC and the ATX loaded particles (pellets) are prepared as per the methods mentioned herein and thereafter are coated with polymers/excipients having other API. Thereafter, the coated particles are delivered as tablets with excipients or filled in capsules with release modifiers and excipients. Another embodiment uses an active coating of the co-drug. In this case, DEC is contained in the core of the tablet and a suspension or solution containing the ATX is sprayed on the tablets via a conventional coating process. In further embodiments, the tablet is enterically coated or coated with an extended release polymer. Alternatively, the ATX is contained in the core of the tablet and a suspension or solution containing the DEC is sprayed on the tablets via a conventional coating process. In further embodiments, the tablet is enterically coated or coated with an extended release polymer

In embodiments, the DEC is at a dose of 100 mg to 250 mg in an enteric capsule that is encapsulated in a larger capsule containing ATX and olive oil or another oil, where the outer capsule is or is not enteric coated and may be a hard capsule or a gel capsule. DEC in higher doses is known to create nausea and the benefit of an enteric coating is to prevent the DEC from releasing in the stomach and reduce or eliminate the nausea side effect.

The foregoing description and accompanying figures illustrate the principles, embodiments, and modes of operation of the present disclosure. However, the present disclosure should not be construed as being limited to the particular embodiments discussed herein. Additional variations of the embodiments discussed herein will be appreciated by those skilled in the art.

Therefore, the embodiments described herein should be regarded as illustrative rather than restrictive. Accordingly, it should be appreciated that variations to those embodiments may be made by those skilled in the art without departing from the scope of the disclosure as defined by the following claims.

It is to be understood that where the claims or specification refer to “a” or “an” element, such reference is not to be construed that there is only one of the element. If the specification or claims refer to “an additional” element, that does not preclude there being more than one of the additional element.

For purposes of the disclosure, the term “at least” followed by a number is used herein to denote the start of a range beginning with that number (which may be a range having an upper limit or no upper limit, depending on the variable being defined). For example, “at least 1” means 1 or more than 1. Terms of approximation, such as “about,” should be interpreted according to their ordinary and customary meanings as used in the associated art unless indicated otherwise. Absent a specific definition and absent ordinary and customary usage in the associated art, such terms should be interpreted to be ±10% of the base value.

When a range is given as “(a first number) to (a second number)” or “(a first number)-(a second number)” this means a range whose lower limit is the first number and whose upper limit is the second number. For example, 25 to 100 or 25-100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

While inventive concepts have been described and illustrated herein by reference to certain embodiments, various changes and further modifications may be made by those of ordinary skill in the art without departing from the spirit of the inventive concept, the scope of which is to be determined by the following claims.

What is claimed is:

1. A method of treating cancer in a patient in need thereof, comprising administering to the patient a therapeutically effective amount of a copper chelator comprising a tetrathiomolybdate (TTM) salt of formula X(MoS₄), and optionally one or more of diethylcarbamazine (DEC) and astaxanthin (ATX), wherein:
X is (2Li)⁺², (2K)⁺², (2Na)⁺², Mg⁺², Ca⁺², or {[N⁺(R¹)(R²)(R³)(R⁴)][N⁺(R⁵), (R⁶)(R⁷)(R⁸)]};
R¹, R², R³, R⁵, R⁶, and R⁷ are independently H, or an optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl; and
R⁴ and R⁸ are absent or independently H, or an optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl;
wherein when R⁴ is absent, R¹ and R² together with N forms an optionally substituted 5- or 6-membered aromatic ring, wherein up to 2 carbon atoms in the ring may be replaced with a heteroatom selected from the group consisting of O, N, and S;
wherein when R⁸ is absent, R⁵ and R⁶ together with N forms an optionally substituted 5- or 6-membered aromatic ring, wherein up to 2 carbon atoms in the ring may be replaced with a heteroatom selected from the group consisting of O, NH, and S;
wherein R¹ and R², R² and R³, or R² and R⁴, together with N optionally forms an optionally substituted cyclic structure;
wherein R⁵ and R⁶, R⁶ and R⁷, or R⁷ and R⁸, together with N optionally forms an optionally substituted cyclic structure;
wherein R⁴ and R⁸ may be joined by a covalent bond;
wherein R¹, R², R³, R⁵, R⁶, and R⁷ are each independently optionally substituted with One or more of OH, oxo, alkyl, alkenyl, alkenyl, NH₂, NHR⁹, N(R⁹)₂, C═N(OH) or OPO₃H₂, wherein R⁹ is each independently alkyl or —C(═O)(O)-alkyl;
wherein R⁴ and R⁸ are each independently optionally substituted with one or more of OH, oxo, alkyl, alkenyl, alkynyl, NH₂, NHR⁹, N(R⁹)₂, —C═N(OH), or —⁺(R¹⁰)₃ wherein is each independently optionally substituted alkyl; and
wherein one or more —CH₂— groups in R¹, R², R³, R⁴, R⁵, R⁶, R⁷, and R⁸ may be replaced with a moiety selected from the group consisting of O, NH, S, S(O), and S(O)₂.

2. The method of claim 1, wherein the copper chelator comprises at least one of ammonium tetrathiomolybdate ((NH₄)₂MoS₄), Bis-choline tetrathiomolybdate (C₁₀H₂₈MoN₂O₂S₄), ammonium trithiomolybdate ((NH₄)₂MoOS₃), or a combination thereof.

3. The method of claim 1, wherein the copper chelator is administered orally.

4. The method of claim 1, wherein the copper chelator is administered orally in a delayed release oral preparation that releases the copper chelator in the gastrointestinal tract after the preparation passes the stomach.

5. The method of claim 1, wherein the copper chelator is administered in combination with a therapeutically effective amount of ATX.

6. The method of claim 1, wherein the copper chelator is administered in combination with a therapeutically effective amount of DEC.

7. The method of claim 1, wherein the copper chelator is administered in combination with a therapeutically effective amount of DEC and ATX.

8. The method of claim 1, wherein the copper chelator is administered in combination with a therapeutically effective amount of one or more selected from the group consisting of: LEAPS peptide heteroconjugate, inhibitors of 5-lipoxygenase enzyme, diethylcarbamazine, Zileuton, inhibitors of LTA4 hydrolase, inhibitors of LT receptors, Sulforaphane, Multikine, Bestatin, tert-Butylhydroquinone, Montelukast, inhibitors of leukotriene B4 receptors BLT1 and/or BLT2, LY293111, BAY-u9773, and combinations thereof.

9. The method of claim 1, wherein the patient is administered TTM in a total dosage in a range of 120 mg-300 mg per day.

10. The method of claim 1, further comprising measuring plasma ceruloplasmin level in the patient, and maintaining or adjusting dosage of the TTM in response thereto.

11. The method of claim 10, wherein when the measured ceruloplasmin level is between 15 mg/dL and 18 mg/dL the TTM dosage is administered to the patient at about 180 mg/day.

12. The method of claim 10, wherein when the measured ceruloplasmin level is below 15 mg/dL the TTM dosage is administered to the patient at about 120 mg/day.

13. The method of claim 10, wherein when the measured ceruloplasmin level is above 20 mg/dL the TTM dosage is administered to the patient at about 240 mg/day-300 mg/day.

14. The method of claim 1, wherein the copper chelator comprising TTM is administered to the patient in a first dosage form, and the one or more of DEC and AXT is administered separately in a second dosage form.

15. A pharmaceutical composition comprising a copper chelator comprising:
a tetrathiomolybdate (TTM) salt of formula X(MoS₄); and
a pharmaceutically acceptable carrier,
wherein:
X is (2Li)⁺², (2K)⁺², (2Na)⁺², Mg⁺², Ca⁺², or {[N⁺(R¹)(R²)(R³)(R⁴)][N⁺(R⁵), (R⁶)(R⁷)(R⁸)]};
R¹, R², R³, R⁵, R⁶, and R⁷ are independently H, or an optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl; and
R⁴ and R⁸ are absent or independently H, or an optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl;
wherein when R⁴ is absent, R¹ and R² together with N forms an optionally substituted 5- or 6-membered aromatic ring, wherein up to 2 carbon atoms in the ring may be replaced with a heteroatom selected from the group consisting of O, N, and S;
wherein when R⁸ is absent, R⁵ and R⁶ together with N forms an optionally substituted 5- or 6-membered aromatic ring, wherein up to 2 carbon atoms in the ring may be replaced with a heteroatom selected from the group consisting of O, NH, and S;
wherein R¹ and R², R² and R³, or R² and R⁴, together with N optionally forms an optionally substituted cyclic structure;
wherein R⁵ and R⁶, R⁶ and R⁷, or R⁷ and R⁸, together with N optionally forms an optionally substituted cyclic structure;
wherein R⁴ and R⁸ may be joined by a covalent bond;
wherein R¹, R², R³, R⁵, R⁶, and R⁷ are each independently optionally substituted with one or more of OH, oxo, alkyl, alkenyl, alkynyl, NH₂, NHR⁹, N(R⁹)₂, —C═N(OH), or OPO₃H₂, wherein R⁹ is each independently alkyl or —C(═O)(O)-alkyl;
wherein R⁴ and R⁸ are each independently optionally substituted with one or more of OH, oxo, alkyl, alkenyl, alkynyl, NH₂, NHR⁹, N(R⁹)₂, —C═N(OH), or ⁺(R¹⁰)₃, wherein R¹⁰ is each independently optionally substituted alkyl; and
wherein one or more —CH₂— groups in R¹, R², R³, R⁴, R⁵, R⁶, R⁷, and R⁸ may be replaced with a moiety selected from the group consisting of O, NH, S, S(O), and S(O)₂, and
wherein the composition is in a delayed release oral form that releases the copper chelator in the gastrointestinal tract after the oral form passes the stomach.

16. The pharmaceutical composition of claim 15, wherein the copper chelator comprises at least one of ammonium tetrathiomolybdate ((NH₄)₂MoS₄), Bis-choline tetrathiomolybdate (C₁₀H₂₈MoN₂O₂S₄), ammonium trithiomolybdate ((NH₄)₂MoOS₃), or a combination thereof.

17. The pharmaceutical composition of claim 15, wherein the composition is a long-acting extended-release oral form.

18. The pharmaceutical composition of claim 15, further comprising one or more of diethylcarbamazine (DEC), astaxanthin (ATX), or a combination thereof.

19. The pharmaceutical composition of claim 15, further comprising DEC.

20. The pharmaceutical composition of claim 15, further comprising ATX.

21. The pharmaceutical composition of claim 15, further comprising DEC and ATX.

22. The pharmaceutical composition of claim 18, wherein the copper chelator comprising TTM is in a first dosage form, and the one or more of DEC, ATX, or a combination thereof is in a separate second dosage form.

23. The pharmaceutical composition of claim 15, further comprising at least one other active agent selected from the group consisting of LEAPS peptide heteroconjugate, inhibitors of 5-lipoxygenase enzyme, diethylcarbamazine, Zileuton, inhibitors of LTA4 hydrolase, inhibitors of LT receptors, Sulforaphane, Multikine, Bestatin, tert-Butylhydroquinone, Montelukast, inhibitors of leukotriene B4 receptors BLT1 and/or BLT2, LY293111, BAY-u9773, and combinations thereof.

24. The pharmaceutical composition of claim 15, wherein the copper chelator is encapsulated inside a first capsule and the first capsule is encapsulated inside a larger second capsule.

25. The pharmaceutical composition of claim 24, wherein the larger second capsule contains a filler, and the first capsule inside the second capsule is isolated from the larger second capsule by the filler such that the first capsule is not in contact with the second capsule.

26. The pharmaceutical composition of claim 25, wherein the filler comprises mesoporous dicalcium phosphate, colloidal silicon dioxide, or a combination thereof.

27. The pharmaceutical composition of claim 24, wherein one or more of the first capsule and the second capsule comprises an enteric coating on an outer surface.

28. The pharmaceutical composition of claim 24, wherein the second capsule contains one or more of DEC and ATX.

29. The pharmaceutical composition of claim 15, wherein the copper chelator is encapsulated by a protective coating comprising an antacid.

30. The pharmaceutical composition of claim 29, wherein the antacid comprises one or more of aluminum hydroxide, calcium carbonate, magnesium carbonate, magnesium hydroxide, magnesium trisilicate, sodium bicarbonate, alginate, or a combination thereof.

31. The pharmaceutical composition of claim 29, further comprising an enteric coating encapsulating the copper chelator and the protective coating.

32. The pharmaceutical composition of claim 18, in a form having a core comprising TTM encapsulated by ATX.

33. The pharmaceutical composition of claim 18, in a form having a core comprising TTM encapsulated by DEC.

34. The pharmaceutical composition of claim 18, in a form having a first core comprising TTM, a second core comprising DEC, the first core and the second core encapsulated by ATX.

35. The pharmaceutical composition of claim 34, wherein the TTM, DEC and ATX are encapsulated by a protective coating comprising an antacid, and further comprising an enteric coating.

36. The pharmaceutical composition of claim 15, further comprising at least one other active agent, a protective coating comprising an antacid encapsulating the copper chelator and the at least one other active agent, and an enteric coating encapsulating the protective coating.

37. The pharmaceutical composition of claim 36, wherein the at least one other active agent is one or more of DEC, ATX, or a combination thereof.

38. The pharmaceutical composition of claim 15, further comprising at least one other active agent, a protective coating comprising an antacid encapsulating the copper chelator, and an enteric coating encapsulating the copper chelator, protective coating, and the at least one other active agent.

39. The pharmaceutical composition of claim 38, wherein the at least one other active agent is one or more of DEC, ATX, or a combination thereof.

40. The pharmaceutical composition of claim 15, wherein the TTM has a dosage in a range of 20-300 mg.

41. The pharmaceutical composition of claim 19, wherein the DEC has a dosage in a range of 100-250 mg.

42. The pharmaceutical composition of claim 20, wherein the ATX has a dosage in a range of 5-30 mg.

43. The pharmaceutical composition of claim 15, wherein at least 50% of the TTM salt has a particle size smaller 44 μm.

44. The pharmaceutical composition of claim 15, wherein at least 90% of the TTM salt has a particle size smaller than 122 μm.

45. The pharmaceutical composition of claim 15, wherein no less than 50% of the TTM salt has a particle size smaller than 122 μm.

46. The pharmaceutical composition of claim 15, wherein the composition is in an extended-release form.

47. The pharmaceutical composition of claim 32, further comprising an enteric coating encapsulating the composition.

48. The pharmaceutical composition of claim 33, further comprising an enteric coating encapsulating the composition.

49. The pharmaceutical composition of claim 34, further comprising an enteric coating encapsulating the composition.

50. A method of treating a cancer patient, as adjuvant therapy in patients undergoing or have undergone radiation therapy, chemotherapy, and/or immunotherapy, or otherwise in need thereof, comprising administering to the patient a therapeutically amount of a copper chelator comprising a tetrathiomolybdate (TTM) salt of formula X(MoS₄), and optionally one or more of diethylcarbamazine (DEC) and astaxanthin (ATX), wherein:
X is (2Li)⁺², (2K)⁺², (2Na)⁺², Mg⁺², Ca⁺², or {[N⁺(R¹)(R²)(R³)(R⁴)][N⁺(R⁵), (R⁶)(R⁷)(R⁸)]};
R¹, R², R³, R⁵, R⁶, and R⁷ are independently H, or an optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl; and
R⁴ and R⁸ are absent or independently H, or an optionally substituted group selected from the group consisting of alkyl, alkenyl, alkynyl, aryl, heteroaryl, cycloalkyl, heterocycloalkyl, aralkyl, alkylaralkyl, heteroalkyl, heteroaralkyl, cycloalkyl alkyl, d heterocycloalkyl alkyl;
wherein when R⁴ is absent, R¹ and R² together with N forms an optionally substituted 5- or 6-membered aromatic ring, wherein up to 2 carbon atoms in the ring may be replaced with a heteroatom selected from the group consisting of O, N, and S;
wherein when R⁸ is absent, R⁵ and R⁶ together with N forms an optionally substituted 5- or 6-membered aromatic ring, wherein up to 2 carbon atoms in the ring may be replaced with a heteroatom selected from the group consisting of O, NH, and S;
wherein R¹ and R², R² and R³, or R² and R⁴, together with N optionally forms an optionally substituted cyclic structure;
wherein R⁵ and R⁶, R⁶ and R⁷, or R⁷ and R⁸, together with N optionally forms an optionally substituted cyclic structure;
wherein R⁴ and R⁸ may be joined by a covalent bond;
wherein R¹, R², R³, R⁵, R⁶, and R⁷ are each independently optionally substituted with one or more of OH, oxo, alkyl, alkenyl, alkynyl, NH₂, NHR⁹, N(R⁹)₂, —C≡N(OH), or OPO₃H₂, wherein R⁹ is each independently alkyl or —C(═O)(O)-alkyl;
wherein R⁴ and R⁸ are each independently optionally substituted with one or more of OH, oxo, alkyl, alkenyl, alkynyl, NH₂, NHR⁹, N(R⁹)₂, —C═N(OH), or ⁺(R¹⁰)₃, wherein R¹⁰ is each independently optionally substituted alkyl; and
wherein one or more —CH₂— groups in R¹, R², R⁴, R⁵, R⁶, R⁷, and R⁸ may be replaced a moiety selected from the group consisting of O, NH, S, S(O), and S(O)₂.

51. A method of manufacturing the pharmaceutical composition of claim 15, wherein the TTM is protected from oxidation during manufacture by an inert gas.

52. A method of manufacturing the pharmaceutical composition of claim 25, wherein the second capsule containing filler and the first capsule inside the second capsule are vibrated so that the inner capsule is positioned so that the outer surface of the inner capsule contacts the filler and does not contact the inner surface of the second capsule.
